(19) United States
(12) Patent Application Publication
(10) Pub. No.: US 2004/0248958 A1
(43) Pub. Date: Dec. 9, 2004

(54) Heterocyclic amide derivatives

(76) Inventors: Graham Holmwood, Wuppertal (DE); Klaus-Gunther Tietjen, Langenfeld (DE); Michael Schindler, Bergisch Gladbach (DE); Christoph Erdelen, Leichlingen (DE); Angelika Lubos-Erdelen, Leichlingen (DE); Ulrike Wachendorff-Neumann, Neuwied (DE); Andreas Turberg, Haan (DE); Olaf Hansen, Leichlingen (DE)

Correspondence Address: BAYER CROPSCIENCE LP, Patent Department, 100 BAYER ROAD, PITTSBURGH, PA, 15205-9741, US

(21) Appl. No.: 10/482,866

(22) PCT Filed: Jun. 24, 2002
(86) PCT No.: PCT/EP02/06940

(30) Foreign Application Priority Data
Jul. 6, 2001 (DE) 101 32 896.6

(51) Int. Cl. A61K 31/4178; A61K 31/4172; C07D 233/66; C 07D 4 3/02

(52) U.S. Cl. 514/397; 514/398; 548/331.1; 548/311.1

(57) ABSTRACT

The present invention relates to novel heterocyclic amide derivatives of the formula

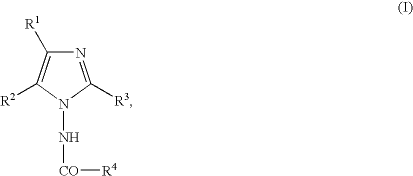

in which R¹, R², R³ and R⁴ are as defined in the disclosure,

to a process for preparing them, and to their use as pesticides.

The present invention relates to novel heterocyclic amide derivatives, to processes for preparing them and to their use as pesticides.

It is known that certain 1-benzylamino-, 1-benzylideneamino- and 1-benzoylaminoimidazoles have pharmacological properties (cf. EP-A-283 857). Use of the substances specified therein as pesticides is, however, not described.

The invention now provides novel compounds of the formula (I)

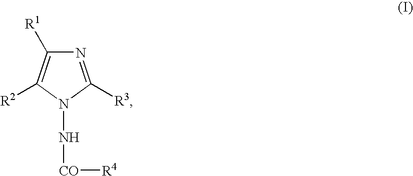

in which

R¹ and R² independently of one another represent hydrogen, alkyl, alkoxyalkyl, alkylthioalkyl, represent optionally substituted cycloalkyl, represent optionally substituted heterocyclyl or, together with the carbon atoms to which they are attached, represent an optionally substituted mono- or bicyclic, carbocyclic or heterocyclic group.

R³ and R⁴ independently of one another represent aryl or hetaryl, each optionally substituted.

It has additionally been found that the novel compounds of the formula (1) are obtained if compounds of the formula (II)

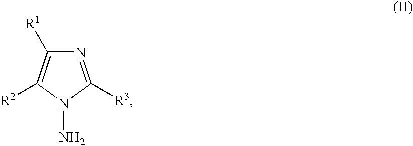

in which

R¹, R² and R³ are as defined above

are reacted with compounds of the formula (III)

R⁵—COR⁴ (III)

in which

R⁴ is as defined above and

R⁵ represents halogen (especially chlorine) or hydroxyl

in the presence of a diluent and an acid acceptor and/or a carboxylic-acid-activating reagent (coupling reagent) and optionally a basic reaction auxiliary.

Finally it has been found that the novel compounds of the formula (1) possess strongly pronounced biological properties and are suitable in particular for controlling animal pests, especially insects, spider mites and nematodes which occur in agriculture, in forestry, in the protection of stored products and of materials, and in the hygiene sector.

A general definition of the compounds of the invention is given by the formula (1).

Preferred substituents and ranges of the radicals set out in the formulae mentioned above and below are elucidated in the following text:

R¹ and R² independently of one another preferably represent hydrogen, C₁-C₆-alkyl, C₁-C₆-alkoxy-C₁-C₃-alkyl, C₁-C₃-alkylthio-C₁-C₃-alkyl, represent in each case optionally C₁-C₃-alkyl-substituted C₃-C₇-cycloalkyl, which may optionally be interrupted by one or two oxygen and/or sulphur atoms, or, together with the carbon atoms to which they are attached, represent optionally C₁-C₃-alkyl-substituted C₃-C₄-alkanediyl, in which two carbon atoms that are not directly adjacent optionally form a further cyclic structure which may optionally be interrupted by one oxygen and/or sulphur atom.

R³ preferably represents phenyl or hetaryl in each case optionally substituted once or twice by identical or different C₁-C₄-alkyl, C₁-C₄-alkoxy, halogen, C₁-C₄-haloalkyl, C₁-C₄-haloalkoxy, cyano, nitro, C₁-C₄-alkylsulfonyl, C₁-C₄-haloalkylsulfonyl, thioamide or tetrazole substituents (for example pyridyl, thienyl, furyl, pyrimidinyl).

R⁴ preferably represents phenyl in each case optionally substituted from one to four times by identical or different C₁-C₄-alkyl, C₁-C₄-alkoxy, C₁-C₄-alkoxy-C₁-C₄-alkoxy, halogen, C₁-C₄-haloalkyl, C₁-C₄-haloalkoxy or cyano substituents, in each case two adjacent carbon atoms optionally forming a five- or six-membered carbocycle which may optionally be interrupted by one or two oxygen atoms, or hetaryl (for example pyridyl, thienyl, furyl, pyrimidinyl).

R¹ and R² independently of one another with particular preference represent hydrogen, C₁-C₅-alkyl, C₁-C₃-alkoxy-C₁-C₃-alkyl, C₁-C₃-alkylthio-C₁-C₃-alkyl, represent C₃-C₆-cycloalkyl or, together with the carbon atoms to which they are attached, represent C₃-C₄-alkanediyl in each case optionally substituted from one to three times by identical or different C₁-C₃-alkyl substituents and in which two carbon atoms not directly adjacent optionally form a further cyclic structure which may optionally be interrupted by an oxygen or sulphur atom.

R³ with particular preference represents phenyl, pyridyl, thienyl, furyl or pyrimidinyl in each case optionally substituted once or twice by identical or different methyl, fluorine, chlorine, bromine, iodine, cyano, nitro, methoxy, trifiluoromethyl, trifluoromethoxy, sulfonylmethyl, sulfonyltrifluoromethyl, thioamide or tetrazole substituents.

R⁴ with particular preference represents phenyl in each case optionally substituted from one to four times by identical or different methyl, ethyl, fluorine, chlorine, bromine, trifluoromethyl, methoxy, trifluoromethoxy, ethoxymethyl ether or cyano substituents, two adjacent carbon atoms optionally forming a five- or six-membered carbocycle which may optionally be interrupted by one or two oxygen atoms.

R¹ with very particular preference represents hydrogen or methyl,

R² with very particular preference represents hydrogen, methyl, ethyl, n- or isopropyl, n-, iso- or s-butyl, cyclopentyl, or cyclohexyl, or

R¹ and R², together with the carbon atoms to which they are attached, with very particular preference represent C₃-C₄-alkanediyl optionally substituted once or twice by methyl or represent C₄-alkanediyl in which two carbon atoms not directly adjacent optionally form a further cyclic structure which may optionally be interrupted by an oxygen atom,

R³ with very particular preference represents phenyl optionally substituted once or twice by identical or different methyl, fluorine, chlorine, bromine, iodine, nitro, cyano, methoxy or trifluoromethyl substituents.

R⁴ with very particular preference represents phenyl in each case optionally substituted from one to four times by identical or different methyl, ethyl, fluorine, chlorine, bromine, trifluoromethyl, methoxy, trifluoromethoxy, 2-methoxyethoxy or cyano substituents, two adjacent carbon atoms optionally forming a five- or six-membered carbocycle which may optionally be interrupted by one or two oxygen atoms.

The definitions of radicals and/or elucidations given above or given in ranges of preference apply to the end products and to the precursors and intermediates correspondingly. These definitions of radicals may be combined as desired with one another, hence including combinations between the respective ranges of preference.

Preference in accordance with the invention is given to the compounds of the formula (I) in which there is a combination of the definitions set out above as being preferred (preferably).

Particular preference in accordance with the invention is given to the compounds of the formula (I) in which there is a combination of the definitions set out above as being particularly preferred (with particular preference).

Very particular preference in accordance with the invention is given to the compounds of the formula (I) in which there is a combination of the definitions set out above as being very particularly preferred (with very particular preference).

In the definitions of radicals set out above and below hydrocarbon radicals, such as alkyl, both alone and in conjunction with heteroatoms, such as alkoxy, are where possible in each case straight-chain or branched.

Using, for example, 1-amino-4,5-dimethyl-2-(3,5-dimethylphenyl)imidazole and 4-ethylbenzoyl chloride as starting materials, the course of reaction in the process of the invention may be depicted by the following formula scheme:

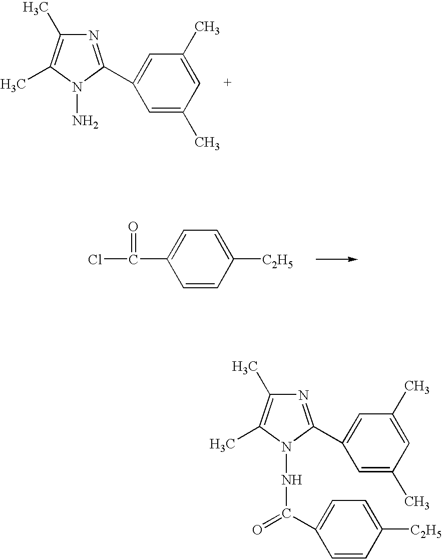

A general definition of the compounds to be used as starting materials for carrying out the process of the invention is given by the formulae (II) and (III).

The compounds of the formula (II) are novel with the exception of 1-amino-2-phenylimidazole, 1-amino-2-(4-chlorophenyl)imidazole, 1-amino-2-(4-methoxy-phenyl)imidazole and 1-amino-2-(3,4-dimethoxyphenyl)imidazole.

They may be obtained by subjecting compounds of the formula (IV)

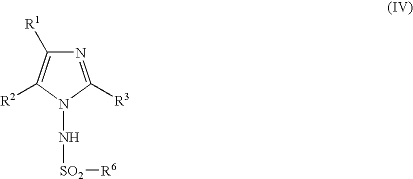

in which

R¹, R² and R³ are as defined above and R⁶ denotes alkyl or optionally substituted phenyl to hydrolytic cleavage with an acid, for example, optionally in the presence of a diluent.

The compounds of the formula (IV) are likewise novel.

They may be obtained by reacting compounds of the formula (V)

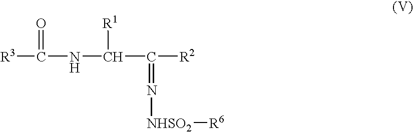

in which R¹, R², R³ and R⁶ are as defined above

with a water-removing agent such as POCl₃, PCl₅ or SOCl₂, for example, optionally in the presence of a diluent.

The compounds of the formula (V) are known in principle and may be prepared in accordance with a process known from the literature (Liebigs Ann. Chem. 1978, 1916).

Some of the compounds of the formula (III) are novel.

The following compounds of the formula (III) are novel:

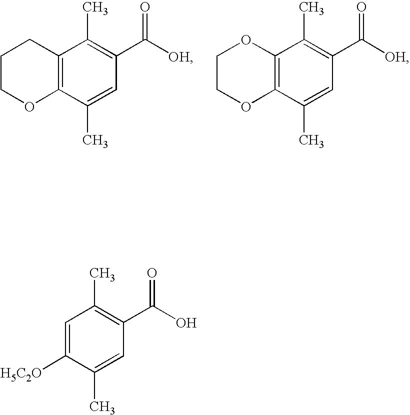

The compounds of the formula (III) may be prepared in accordance with the Preparation Examples (III-1, III-2 and III-3).

The process of the invention is preferably carried out using diluents. Suitable diluents include virtually all inert organic solvents. These include preferably aliphatic and aromatic, optionally halogenated hydrocarbons such as pentane, hexane, heptane, cyclohexane, petroleum ether, benzine, ligroin, benzene, toluene, xylene, methylene chloride, ethylene chloride, chloroform, carbon tetrachloride, chlorobenzene and o-dichlorobenzene, ethers such as diethyl and dibutyl ether, glycol dimethyl ether and diglycol dimethyl ether, tetrahydrofuran and dioxane, ketones such as acetone, methyl ethyl ketone, methyl isopropyl ketone or methyl isobutyl ketone, esters such as methyl or ethyl acetate, nitrites such as acetonitrile or propionitrile, for example, amides such as dimethylformamide, dimethylacetamide and N-methylpyrrolidone, for example, and also dimethyl sulfoxide, tetramethylene sulfone or hexamethylphosphoramide.

As acid acceptors in the process of the invention it is possible to use all acid binders which can commonly be used for such reactions. Those suitable include preferably alkali metal and alkaline earth metal hydrides, such as lithium, sodium, potassium or calcium hydride, alkali metal or alkaline earth metal hydroxides, such as lithium, sodium, potassium or calcium hydroxide, alkali metal or alkaline earth metal carbonates or bicarbonates, such as sodium or potassium carbonates or bicarbonate or calcium carbonate, alkali metal acetates, such as sodium or potassium acetate, alkali metal alkoxides, such as sodium or potassium tert-butoxide, and also basic nitrogen compounds, such as trimethylamine, triethylamine, tripropylamine, tributylamine, diisobutylamine, dicyclohexylamine, ethyldiisopropylamine, ethyldicyclohexylamine, N,N-dimethylbenzylamine, N,N-dimethylaniline, pyridine, 2-methyl-, 3-methyl-, 4-methyl-, 2,4-dimethyl-, 2,6-dimethyl-, 2-ethyl-, 4-ethyl- and 5-ethyl-2-methyl-pyridine, 1,5-diazabicyclo[4.3.0]non-5-ene (DBN), 1,8-diazabicyclo[5.4.0]undec-7-ene (DBU) or 1,4-diazabicyclo[2.2.2]octane (DABCO).

As carboxylic-acid-activating reagent (coupling reagent) use is made in the process of the invention of all those suitable for preparing an amide linkage (cf. e.g.: Houben-Weyl, Methoden der organischen Chemie, Volume 15/2; Bodanszky et al., Peptide Synthesis 2nd ed. (Wiley & Sons, New York 1976) or Gross, Meienhofer, The Peptides: Analysis Synthesis, Biology (Academic Press, New York 1979). It is preferred to employ the following methods: active ester method with pentachlorophenol (Pcp) and pentafluorophenol (Pfp), N-hydroxysuccinimide, N-hydroxy-5-norbornene-2,3-dicarboxamide (HONB), 1-hydroxybenzotriazole (HOBt) or 3-hydroxy-4-oxo-3,4-dihydro-1,2,3-benzotriazine as alcohol component, coupling with carbodiimides such as dicyclohexylcarbodiimide (DCC) by the DCC additive process, or with n-propanephosphonic anhydride (PPA) and mixed-anhydride method using pivaloyl chloride, ethyl (EEDQ) and isobutyl (IIDQ) chloroformate or coupling with phosphonium reagents, such as benzotriazol-1-yloxytris(dimethylaminophosphonium) hexafluorophosphate (BOP), bis(2-oxo-3-oxazolidinyl)phosphonium chloride (BOP-Cl), or with phosphonic ester reagents, such as diethyl cyanophosphonate (DEPC) and diphenylphosphoryl azide (DPPA) or uronium reagents, such as 2-(1H-benzotriazol-1-yl)-1,1,3,3-tetramethyluronium tetrafluoroborate (TBTU).

As basic reaction auxiliaries for carrying out the process of the invention it is possible to use all suitable acid binders such as amines, especially tertiary amines, and also alkali metal compounds and alkaline earth metal compounds.

Examples thereof that may be mentioned include the hydroxides, oxides and carbonates of lithium, sodium, potassium, magnesium, calcium and barium, and also other basic compounds such as amidine bases or guanidine bases such as 7-methyl-1,5,7-triazabicyclo[4.4.0]dec-5-ene (MTBD), diazabicyclo[4.3.0]nonene (DBN), diazabicyclo-[2.2.2]octane (DABCO), 1,8-diazabicyclo[5.4.0]undecene (DBU), cyclohexyltetra-butylguanidine (CyTBG), cyclohexyltetramethylguanidine (CyTMG), N,N,N,N-tetramethyl-1,8-naphthalenediamine, pentamethylpiperidine, tertiary amines such as triethylamine, trimethylamine, tribenzylamine, triisopropylamine, tributylamine, tribenzylamine, tricyclohexylamine, triamylamine, trihexylamine, N,N-dimethylaniline, N,N-dimethyltoluidine, N,N-dimethyl-p-aminopyridine, N-methylpyrrolidine, N-methylpiperidine, N-methylimidazole, N-methylpyrrole, N-methylmorpholine, N-methylhexamethyleneimine, pyridine, 4-pyrrolidinopyridine, 4-dimethylaminopyridine, quinoline, α-picoline, isoquinoline, pyrimidine, acridine, N,N,N′,N′-tetramethylenediamine, N,N,N′,N′-tetraethylenediamine, quinoxaline, N-propyldiisopropylamine, N-ethyldiisopropylamine, N,N′-dimethylcyclohexylamine, 2,6-lutidine, 2,4-lutidine or triethylenediamine.

The reaction temperatures in the process of the invention may be varied within a relatively large range. It is normal to operate at temperatures between −20° C. and 80° C., preferably at temperatures between 0° C. and 50° C.

The process of the invention is generally carried out under atmospheric pressure. It is, however, also possible to operate under increased or reduced pressure.

For carrying out the process of the invention the particular requisite starting materials are used generally in approximately equimolar amounts. It is, however, also possible to use one of the two components used in each case in a relatively large excess. It may also be advantageous to operate with 2 or more equivalents of the compound of the formula (III). In this case the product is predominantly a bisbenzoylated derivative, which in a second step is hydrolytically cleaved to the monobenzoylated derivative. Workup in the processes of the invention takes place in each case in accordance with customary methods (cf. Preparation Examples).

It has also been found that the novel compounds of the formula (I) are obtained if compounds of the formula (VI)

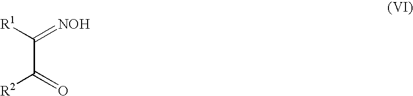

in which

R¹ and R² are as defined above

are reacted with compounds of the formula (VII)

R³CHO (VII)

in which

R³ is as defined above and

compounds of the formula (VII)

R⁴CO NH NH₂ (VIII)

in which

R⁴ is as defined above

in the presence of a diluent at temperatures between 20° C. and 160° C. and the resulting N-oxide derivative is reacted with zinc in glacial acetic acid at temperatures between 20° C. and 100° C. (Z. Chem. 1971, 11, 10) or titanium tetrachloride and sodium iodide in the presence of a diluent at temperatures between 0° C. and 60° C. (Chem. Ber. 1990, 123, 647) or trialkyl phosphite in the presence of a diluent at temperatures between 0° C. and 80° C. (JCS, 1962, 1917) or phosphorus trichloride in the presence of a diluent at temperatures between 10° C. and 100° C. (Tet. 1999, 55, 1441) or DDQ in the presence of a diluent at temperatures between 10° C. and 100° C. (Tet. 1999, 55, 1441) or TCNQ/TCNQF4 in the presence of a diluent at temperatures between 0° C. and 80° C. (J. Chem. Res. (S) 1999, 620).

The compounds of the formulae (VI), (VII) and (VIII) are known in principle and in many cases are indeed available commercially. Compounds of the formula (VI) may be obtained, for example, in accordance with Org. Syn. Coll. Vol. II, 204; compounds of the formula (VIII), for example, in accordance with Pharmazie 1996, 51, 83.

The active compounds are suitable for controlling animal pests, in particular insects, arachnids and nematodes, which are encountered in agriculture, in forestry, in the protection of stored products and of materials, and in the hygiene sector, and have good plant tolerance and favourable toxicity to warm-blooded animals. They may preferably be employed as plant protection agents. They are active against normally sensitive and resistant species and against all or some stages of development. The above-mentioned pests include:

From the order of the Isopoda, for example, Oniscus asellus, Armadillidium vulgare and Porcellio scaber.

From the order of the Diplopoda, for example, Blaniulus guttulatus.

From the order of the Chilopoda, for example, Geophilus carpophagus and Scutigera spp.

From the order of the Symphyla, for example, Scutigerella immaculate.

From the order of the Thysanura, for example, Lepisma saccharina.

From the order of the Collembola, for example, Onychiurus armatus.

From the order of the Orthoptera, for example, Acheta domesticus, Gryllotalpa spp., Locusta migratoria migratorioides, Melanoplus spp. and Schistocerca gregaria.

From the order of the Blattaria, for example, Blatta orientalis, Periplaneta americana, Leucophaea maderae and Blattella gemmanica.

From the order of the Dermaptera, for example, Forficula auricularia.

From the order of the Isoptera ,for example, Reticulitermes spp.

From the order of the Phthiraptera, for example, Pediculus humanus corporis, Haematopinus spp., Linognathus spp., Trichoidectes spp. and Damalinia spp.

From the order of the Thysanoptera ,for example, Hercinothrips femoralis, Thrips tabaci, Thrips palmi and Frankliniella accidentalis.

From the order of the Heteroptera, for example, Eurygaster spp., Dysdercus intermedius, Piesma quadrata, Cimex lectularius, Rhodnius prolixus and Triatoma spp.

From the order of the Homoptera ,for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp. and Psylla spp.

From the order of the Lepidoptera ,for example, Pectinophora gossypiella, Bupalus piniarius, Cheimatobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, Cnaphalocerus spp. and Oulema oryzae.

From the order of the Coleoptera ,for example, Anobium punctatum, Rhizopertha dorninica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica and Lissorhoptrus oryzophilus.

From the order of the Hymenoptera ,for example, Diprion spp., Hoplocampa spp., Lasius spp., Monomorium pharaonis and Vespa spp.

From the order of the Diptera ,for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp. and Liriomyza spp.

From the order of the Siphonaptera ,for example, Xenopsylla cheopis and Ceratophyllus spp.

From the class of the Arachnida, for example, Scorpio maurus, Latrodectus mactans, Acarus siro, Argas spp., Omithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp. and Brevipalpus spp.

The phytoparasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp. and Bursaphelenchus spp.

If appropriate, the compounds according to the invention can, at certain concentrations or application rates, also be used as herbicides or microbicides, for example as fungicides, antimycotics and bactericides. If appropriate, they can also be employed as intermediates or precursors for the synthesis of other active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and recombinant methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offshoots and seeds.

The treatment according to the invention of the plants and plant parts with the active compounds is carried out directly or by allowing the compounds to act on the surroundings, environment or storage space by the customary treatment methods, for example by immersion, spraying, evaporation, fogging, scattering, painting on and, in the case of propagation material, in particular in the case of seeds, also by applying one or more coats.

The active compounds can be converted into the customary formulations, such as solutions, emulsions, wettable powders, suspensions, powders, dusts, pastes, soluble powders, granules, suspension-emulsion concentrates, natural and synthetic materials impregnated with active compound and microencapsulations in polymeric substances.

These formulations are produced in a known manner, for example by mixing the active compounds with extenders, that is to say liquid solvents and/or solid carriers, optionally with the use of surfactants, that is to say emulsifiers and/or dispersants and/or foam-formers.

If the extender used is water, it is also possible to employ for example organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethylformamide and dimethyl sulphoxide, and also water.

Suitable solid carriers are:

for example ammonium salts and ground natural minerals such as kaolins, clays, talc, chalk, quartz, attapulgite, montmorillonite or diatomaceous earth, and ground synthetic minerals, such as highly disperse silica, alumina and silicates; suitable solid carriers for granules are: for example crushed and fractionated natural rocks such as calcite, marble, pumice, sepiolite and dolomite, and also synthetic granules of inorganic and organic meals, and granules of organic material such as sawdust, coconut shells, maize cobs and tobacco stalks; suitable emulsifiers and/or foam-formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates; suitable dispersants are: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations. Other additives can be mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95% by weight of active compound, preferably between 0.5 and 90%.

The active compounds according to the invention can be used as such or in their formulations, including mixtures with known fungicides, bactericides, acaricides, nematicides or insecticides, so as, for example, to broaden the spectrum of action or prevent resistances developing. In many cases here, synergistic effects are obtained, i.e. the activity of the mixture is greater than the activity of the individual components.

Suitable co-components are, for example, the following compounds:

Fungicides:

aldimorph, ampropylfos, ampropylfos-potassium, andoprim, anilazine, azaconazole, azoxystrobin,

benalaxyl, benodanil, benomyl, benzamacril, benzamacryl-isobutyl, bialaphos, binapacryl, biphenyl, bitertanol, blasticidin-S, bromuconazole, bupirimate, buthiobate,

calcium polysulphide, carpropamid, capsimycin, captafol, captan, carbendazim, carboxin, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram,

debacarb, dichlorophen, diclobutrazole, diclofluanid, diclomezine, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon,

edifenphos, epoxiconazole, etaconazole, ethirimol, etridiazole,

famoxadon, fenapanil, fenarimol, fenbuconazole, fenfuram, fenhexamid, fenitropan, fenpiclonil, fenpropidin, fenpropimorph, fentin acetate, fentin hydroxide, ferbam, ferimzone, fluazinam, flumetover, fluoromide, fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox,

guazatine,

hexachlorobenzene, hexaconazole, hymexazole,

imazalil, irnibenconazole, iminoctadine, iminoctadine albesilate, iminoctadine triacetate, iodocarb, ipconazole, iprobenfos (IBP), iprodione, iprovalicarb, irumamycin, isoprothiolane, isovaledione,

kasugamycin, kresoxim-methyl, copper preparations, such as: copper hydroxide, copper naphthenate, copper oxychloride, copper sulphate, copper oxide, oxine-copper and Bordeaux mixture,

mancopper, mancozeb, maneb, meferimzone, mepanipyrim, mepronil, metalaxyl, metconazole, methasulfocarb, methfuroxam, metiram, metomeclam, metsulfovax, mildiomycin, myclobutanil, myclozolin,

nickel dimethyldithiocarbamate, nitrothal-isopropyl, nuarimol,

ofurace, oxadixyl, oxamocarb, oxolinic acid, oxycarboxim, oxyfenthiin,

paclobutrazole, pefurazoate, penconazole, pencycuron, phosdiphen, picoxystrobin, pimaricin, piperalin, polyoxin, polyoxorim, probenazole, prochloraz, procymidone, propamocarb, propanosine-sodium, propiconazole, propineb, pyraclostrobin, pyrazophos, pyrifenox, pyrimethanil, pyroquilon, pyroxyfur,

quinconazole, quintozene (PCNB), quinoxyfen,

sulphur and sulphur preparations, spiroxamine,

tebuconazole, tecloftalam, tecnazene, tetcyclacis, tetraconazole, thiabendazole, thicyofen, thifluzamide, thiophanate-methyl, thiram, tioxymid, toiclofos-methyl, tolylfluanid, triadimefon, triadimenol, triazbutil, triazoxide, trichiamide, tricyclazole, tridemorph, trifloxystrobin, triflumizole, triforine, triticonazole,

uniconazole,

validamycin A, vinclozolin, viniconazole,

zarilamide, zineb, ziram and also Dagger G,

OK-8705,

OK-8801,

α-(1,1-dimethylethyl)-β-(2-phenoxyethyl)-1H-1,2,4-triazole-1-ethanol,

α-(2,4-dichlorophenyl)-α-fluoro-b-propyl-1H-1,2,4-triazole-1-ethanol,

α-(2,4-dichlorophenyl)-β-methoxy-a-methyl-1H-1,2,4-triazole-1-ethanol,

α-(5-methyl-1,3-dioxan-5-yl)-β-[[4-(trifluoromethyl)-phenyl]-methylene]-1H-1,2,4-triazole-1-ethanol,

(5RS)6RS)-6-hydroxy-2,2,7,7-tetramethyl-5-(1H-1,2,4-triazol-1-yl)-3-octanone,

(E)-α-(methoxyimino)-N-methyl-2-phenoxy-phenylacetamide,

1-(2,4-dichlorophenyl)-2-(1H-1,2,4-triazol-1-yl)-ethanone-O-(phenylmethyl)-oxime,

1-(2-methyl-1-naphthalenyl)-1H-pyrrole-2,5-dione,

1-(3,5-dichlorophenyl)-3-(2-propenyl)-2,5-pyrrolidinedione,

1-[(diiodomethyl)-sulphonyl]4-methyl-benzene,

1-[[2-(2,4-dichlorophenyl)-1,3-dioxolan-2-yl]-methyl]-1H-imidazole,

1-[[2-(4-chlorophenyl)-3-phenyloxiranyl]-methyl]-1H-1,2,4-triazole,

1-[1-[2-[(2,4-dichlorophenyl)-methoxy]-phenyl]-ethenyl]-1H-imidazole,

1-methyl-5-nonyl-2-(phenylmethyl)-3-pyrrolidinole,

2′,6′-dibromo-2-methyl-4′-trifluoromethoxy-4′-trifluoromethyl-1,3-thiazole-5-carboxanilide,

2,6-dichloro-5-(methylthio)₄-pyrimidinyl-thiocyanate,

2,6-dichloro-N-(4-trifluoromethylbenzyl)-benzamide,

2,6-dichloro-N-[[4-(trifluoromethyl)-phenyl]-methyl]-benzamide,

2-(2,3,3-triiodo-2-propenyl)-2H-tetrazole,

2-[(1-methylethyl)-sulphonyl]-5-(trichloromethyl)-1,3,4-thiadiazole,

2-[[6-deoxy-4-O-(4-O-methyl-β-D-glycopyranosyl)-a-D-glucopyranosyl]-amino]4-methoxy-1H-pyrrolo[2,3-d]pyrimidine-5-carbonitrile,

2-aminobutane,

2-bromo-2-(bromomethyl)-pentanedinitrile,

2-chloro-N-(2,3-dihydro-1,1,3-trimethyl-1H-inden-4-yl)-3-pyridinecarboxamide,

2-chloro-N-(2,6-dimethylphenyl)-N-(i sothiocyanatomethyl)-acetamide,

2-phenylphenol (OPP),

3,4-dichloro-1-[4-(difluoromethoxy)-phenyl]-1H-pyrrole-2,5-dione,

3,5-dichloro-N-[cyano[(1-methyl-2-propynyl)-oxy]-methyl]-benzamide,

3-(1,1-dimethylpropyl)-1-oxo-1H-indene-2-carbonitrile,

3-[2-(4-chlorophenyl)-5-ethoxy-3-isoxazolidinyl]-pyridine,

4-chloro-2-cyano-N,N-dimethyl-5-(4-methylphenyl)-1H-imidazole-1-sulphonamide,

4-methyl-tetrazolo[1,5-a]quinazolin-5(4H)-one,

8-hydroxyquinoline sulphate,

9H-xanthene-2-[(phenylamino)-carbonyl]-9-carboxylic hydrazide,

bis-(1-methylethyl)-3-methyl-4-[(3-methylbenzoyl)-oxy]-2,5-thiophenedicarboxylate,

cis-1-(4-chlorophenyl)-2-(1H-1,2,4-triazol-1-yl)-cycloheptanol,

cis-4-[3-[4-(1,1-dimethylpropyl)-phenyl]-2-methylpropyl]-2,6-dimethyl-morpholine hydrochloride,

ethyl [(4-chlorophenyl)-azo]-cyanoacetate,

potassium bicarbonate,

methanetetrathiol-sodium salt,

methyl 1-(2,3-dihydro-2,2-dimethyl-1H-inden-1-yl)-1H-imidazole-5-carboxylate,

methyl N-(2,6-dimethylphenyl)-N-(5-isoxazolylcarbonyl)-DL-alaninate,

methyl N-(chloroacetyl)-N-(2,6-dimethylphenyl)-DL-alaninate,

N-(2,6-dimethylphenyl)-2-methoxy-N-(tetrahydro-2-oxo-3-furanyl)-acetamide,

N-(2,6-dimethylphenyl)-2-methoxy-N-(tetrahydro-2-oxo-3-thienyl)-acetamide,

N-(2-chloro-4-nitrophenyl)₄-methyl-3-nitro-benzenesulphonamide,

N-(4-cyclohexylphenyl)-1,4,5,6-tetrahydro-2-pyrimidineamine,

N-(4-hexylphenyl)-1,4,5,6-tetrahydro-2-pyrimidineamine,

N-(5-chloro-2-methylphenyl)-2-methoxy-N-(2-oxo-3-oxazolidinyl)-acetamide,

N-(6-methoxy-3-pyridinyl)-cyclopropanecarboxamide,

N-[2,2,2-trichloro-1-[(chloroacetyl)-aniino]-ethyl]-benzamide,

N-[3-chloro-4,5-bis-(2-propinyloxy)-phenyl]-N′-methoxy-methaneimidamide,

N-formyl-N-hydroxy-DL-alanine-sodium salt,

O,O-diethyl [2-(dipropylamino)-2-oxoethyl]-ethylphosphoramidothioate,

O-methyl S-phenyl phenylpropylphosphoramidothioate,

S-methyl 1,2,3-benzothiadiazole-7-carbothioate,

spiro[2H]-1-benzopyran-2,1′ (3′H)-isobenzofuran]-3′-one;

4-[3,4-dimethoxyphenyl-3-(4-fluorophenyl)-acryloyl]-morpholine.

Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

Insecticides/acaricides/nematicides:

abamectin, acephate, acetamiprid, acrinathrin, alanycarb, aldicarb, aldoxycarb, alpha-cypermethrin, alphamethrin, amitraz, avermectin, AZ 60541, azadirachtin, azamethiphos, azinphos A, azinphos M, azocyclotin,

Bacillus popilliae, Bacillus sphaericus, Bacillus subtilis, Bacillus thuringiensis, baculoviruses, Beauveria bassiana, Beauveria tenella, bendiocarb, benfuracarb, bensultap, benzoximate, betacyfluthrin, bifenazate, bifenthrin, bioethanomethrin, biopermethrin, bistrifluron, BPMC, bromophos A, bufencarb, buprofezin, butathiofos, butocarboxim, butylpyridaben,

cadusafos, carbaryl, carbofuran, carbophenothion, carbosulfan, cartap, chloethocarb, chlorethoxyfos, chlorfenapyr, chlorfenvinphos, chlorfluazuron, chlormephos, chlorpyrifos, chlorpyrifos M, chlovaporthrin, chromafenozide, cis-resmethrin, cispermethrin, clocythrin, cloethocarb, clofentezine, clothianidin, cyanophos, cycloprene, cycloprothrin, cyfluthrin, cyhalothrin, cyhexatin, cypermethrin, cyromazine,

deltamethrin, demeton M, demeton S, demeton-S-methyl, diafenthiuron, diazinon, dichlorvos, dicofol, diflubenzuron, dimethoate, dimethylvinphos, diofenolan, disulfoton, docusat-sodium, dofenapyn,

eflusilanate, emamectin, empenthrin, endosulfan, Entomopfthora spp., esfenvalerate, ethiofencarb, ethion, ethoprophos, etofenprox, etoxazole, etrimfos,

fenamiphos, fenazaquin, fenbutatin oxide, fenitrothion, fenothiocarb, fenoxacrim, fenoxycarb, fenpropathrin, fenpyrad, fenpyrithrin, fenpyroximate, fenvalerate, fipronil, fluazuron, flubrocythrinate, flucycloxuron, flucythrinate, flufenoxuron, flumethrin, flutenzine, fluvalinate, fonophos, fosmethilan, fosthiazate, fubfenprox, furathiocarb,

granulosis viruses,

halofenozide, HCH, heptenophos, hexaflumuron, hexythiazox, hydroprene,

imidacloprid, indoxacarb, isazofos, isofenphos, isoxathion, ivernectin,

nuclear polyhedrosis viruses,

lambda-cyhalothrin, lufenuron,

malathion, mecarbam, metaldehyde, methamidophos, Metharhizium anisopliae, Metharhizium flavoviride, methidathion, methiocarb, methoprene, methomyl, methoxyfenozide, metolcarb, metoxadiazone, mevinphos, milbemectin, milbemycin, monocrotophos,

naled, nitenpyram, nithiazine, novaluron,

omethoate, oxamyl, oxydemethon M,

Paecilomyces fumosoroseus, parathion A, parathion M, permethrin, phenthoate, phorate, phosalone, phosmet, phosphamidon, phoxim, pirimicarb, pirimiphos A, pirimiphos M, profenofos, promecarb, propargite, propoxur, prothiofos, prothoate, pymetrozine, pyraclofos, pyresmethrin, pyrethrum, pyridaben, pyridathion, pyrimidifen, pyriproxyfen,

quinalphos,

ribavirin,

salithion, sebufos, silafluofen, spinosad, spirodiclofen, sulfotep, suiprofos,

tau-fluvalinate, tebufenozide, tebufenpyrad, tebupirimiphos, teflubenzuron, tefluthrin, temephos, temivinphos, terbufos, tetrachlorvinphos, tetradifon, theta-cypermethrin, thiacloprid, thiamethoxam, thiapronil, thiatriphos, thiocyclam hydrogen oxalate, thiodicarb, thiofanox, thuringiensin, tralocythrin, tralomethrin, triarathene, triazamate, triazophos, triazuron, trichlophenidine, trichlorfon, triflumuron, trimethacarb,

vamidothion, vaniliprole, Verticillium lecanii,

YI 5302,

zeta-cypermethrin, zolaprofos,

(1R-cis)-[5-(phenylmethyl)-3-furanyl]-methyl-3-[(dihydro-2-oxo-3(2H)-furanylidene)-methyl]-2,2-dimethylcyclopropanecarboxylate,

(3-phenoxyphenyl)-methyl-2,2,3,3-tetramethylcyclopropanecarboxylate,

1-[(2-chloro-5-thiazolyl)methyl]tetrahydro-3,5-dimethyl-N-nitro-1,3,5-triazine-2(1H)-imine,

2-(2-chloro-6-fluorophenyl)-4-[4-(1,1-dimethylethyl)phenyl]4,5-dihydro-oxazole,

2-(acetyloxy)-3-dodecyl-1,4-naphthalenedione,

2-chloro-N-[[[4-(1-phenylethoxy)-phenyl]-amino]-carbonyl]-benzamide,

2-chloro-N-[[[4-(2,2-dichloro-1,1-difluoroethoxy)-phenyl]-amino]-carbonyl]-benzamide,

3-methylphenyl propylcarbamate,

4-[4-(4-ethoxyphenyl)-4-methylpentyl]-1-fluoro-2-phenoxy-benzene,

4-chloro-2-(1,1-dimethylethyl)-5-[[2-(2,6-dimethyl-4-phenoxyphenoxy)ethyl]thio]-3(2H)-pyridazinone,

4-chloro-2-(2-chloro-2-methylpropyl)-5-[(6-iodo-3-pyridinyl)methoxy]-3(2H)-pyridazinone,

4-chloro-5-[(6-chloro-3-pyridinyl)methoxy]-2-(3,4-dichlorophenyl)-3(2H)-pyridazinone,

Bacillus thuringiensis strain EG-2348,

2-benzoyl-1-(1,1-dimethylethyl)-hydrazinobenzoic acid,

2,2-dimethyl-3-(2,4-dichlorophenyl)-2-oxo-1-oxaspiro[4.5]dec-3-en4-yl butanoate,

[3-[(6-chloro-3-pyridinyl)methyl]-2-thiazolidinylidene]-cyanamide,

dihydro-2-(nitromethylene)-2H-1,3-thiazine-3(4H)-carboxaldehyde,

ethyl [2-[[1,6-dihydro-6-oxo-1-(phenylmethyl)₄-pyridazinyl]oxy]ethyl]-carbamate,

N-(3,4,4-trifluoro-1-oxo-3-butenyl)-glycine,

N-(4-chlorophenyl)-3-[4-(difluoromethoxy)phenyl]-4,5-dihydro-4-phenyl-1H-pyrazole-1-carboxamide,

N-[(2-chloro-5-thiazolyl)methyl]-N′-methyl-N″-nitro-guanidine,

N-methyl-N′-(1-methyl-2-propenyl)-1,2-hydrazinedicarbothioamide,

N-methyl-N′-2-propenyl-1,2-hydrazinedicarbothioamide,

O,O-diethyl [2-(dipropylamino)-2-oxoethyl]-ethylphosphoramidothioate,

N-cyanomethyl-4-trifluoromethyl-nicotinamide,

3,5-dichloro-1-(3,3-dichloro-2-propenyloxy)-4-[3-(5-trifluoromethylpyridin-2-yloxy)-propoxy]-benzene.

It is also possible to admix other known active compounds, such as herbicides, fertilizers and growth regulators.

When used as insecticides, the active compounds according to the invention can furthermore be present in their commercially available formulations and in the use forms, prepared from these formulations, in a mixture with synergistic agents.

Synergistic agents are compounds which increase the action of the active compounds, without it being necessary for the synergistic agent added to be active itself.

The active compound content of the use forms prepared from the commercially available formulations can vary within wide limits. The active compound concentration of the use forms can be from 0.0000001 to 95% by weight of active compound, preferably between 0.0001 and 1% by weight.

The compounds are employed in a customary manner appropriate for the use forms.

When used against hygiene pests and pests of stored products, the active compound is distinguished by an excellent residual action on wood and clay as well as a good stability to alkali on limed substrates.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof are treated. The term “parts” or “parts of plants” or “plant parts” has been explained above.

Particularly preferably, plants of the plant cultivars which are in each case commercially available or in use are treated according to the invention. Plant cultivars are to be understood as meaning plants having new properties (“traits”) which have been obtained by conventional breeding, by mutagenesis or by recombinant DNA techniques. These can be varieties, bio- and genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions to be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which extend beyond the effects which were actually to be expected.

The preferred transgenic plants or plant cultivars (i.e. those obtained by genetic engineering) which are to be treated according to the invention include all plants which, in the genetic modification, received genetic material which imparted particular advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grapevines), and particular emphasis is given to maize, soya beans, potatoes, cotton and oilseed rape. Traits that are emphasized are in particular increased defence of the plants against insects by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (hereinbelow referred to as “Bt plants”). Traits that are also particularly emphasized are the increased defence of plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combinations with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these or still to be developed genetic traits, which plants will be developed and/or marketed in the future.

The plants listed can be treated according to the invention in a particularly advantageous manner with the compounds of the general formula I or the active compound mixtures according to the invention. The preferred ranges stated above for the active compounds or mixtures also apply to the treatment of these plants.

Particular emphasis is given to the treatment of plants with the compounds or the mixtures specifically mentioned in the present text.

The active compounds according to the invention act not only against plant, hygiene and stored product pests, but also in the veterinary medicine sector against animal parasites (ectoparasites), such as hard ticks, soft ticks, mange mites, leaf mites, flies (biting and licking), parasitic fly larvae, lice, hair lice, feather lice and fleas. These parasites include:

From the order of the Anoplurida ,for example, Haematopinus spp., Linognathus spp., Pediculus spp., Phtirus spp. and Solenopotes spp.

From the order of the Mallophagida and the suborders Amblycerina and Ischnocerina, for example, Trimenopon spp., Menopon spp., Trinoton spp., Bovicola spp., Werneckiella spp., Lepikentron spp., Damalina spp., Trichodectes spp. and Felicola spp.

From the order of the Diptera andthe suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

From the order of the Siphonapterida ,for example, Pulex spp., Ctenocephalides spp., Xenopsylla spp. and Ceratophyllus spp.

From the order of the Heteropterida ,for example, Cimex spp., Triatoma spp., Rhodnius spp. and Panstrongylus spp.

From the order of the Blattarida ,for example, Blatta orientalis, Periplaneta americana, Blattella germanica and Supella spp.

From the subclass of the Acaria (Acarida)and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

From the order of the Actinedida (Prostigmata)and Acaridida (Astigmata),for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The active compounds of the formula (I) according to the invention are also suitable for controlling arthropods which infest agricultural productive livestock, such as, for example, cattle, sheep, goats, horses, pigs, donkeys, camels, buffalo, rabbits, chickens, turkeys, ducks, geese and bees, other pets, such as, for example, dogs, cats, caged birds and aquarium fish, and also so-called test animals, such as, for example, hamsters, guinea pigs, rats and mice. By controlling these arthropods, cases of death and reduction in productivity (for meat, milk, wool, hides, eggs, honey etc.) should be diminished, so that more economic and easier animal husbandry is possible by use of the active compounds according to the invention.

The active compounds according to the invention are used in the veterinary sector in a known manner by enteral administration in the form of, for example, tablets, capsules, potions, drenches, granules, pastes, boluses, the feed-through process and suppositories, by parenteral administration, such as, for example, by injections (intramuscular, subcutaneous, intravenous, intraperitoneal and the like), implants, by nasal administration, by dermal use in the form, for example, of dipping or bathing, spraying, pouring on and spotting on, washing and powdering, and also with the aid of moulded articles containing the active compound, such as collars, ear marks, tail marks, limb bands, halters, marking devices and the like.

When used for cattle, poultry, pets and the like, the active compounds of the formula (I) can be used as formulations (for example powders, emulsions, free-flowing compositions), which comprise the active compounds in an amount of 1 to 80% by weight, directly or after 100 to 10 000-fold dilution, or they can be used as a chemical bath.

It has furthermore been found that the compounds according to the invention have a strong insecticidal action against insects which destroy industrial materials.

The following insects may be mentioned as examples and as preferences—but without a limitation:

Beetles, such as Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec., Tryptodendron spec., Apate monachus, Bostrychus capucins, Heterobostrychus brunneus, Sinoxylon spec. and Dinoderus minutus.

Hymenopterons, such as

Sirex juvencus, Urocerus gigas, Urocerus gigas taignus and Urocerus augur.

Termites, such as

Kalotermes flavicollis, Cryptotermes brevis, Heteroternes indicola, Reticulitermes flavipes, Reticulitermes santonensis, Reticulitermes lucifugus, Mastotermes darwiniensis, Zootermopsis nevadensis and Coptotermes formosanus.

Bristletails, such as Lepisma saccharina.

Industrial materials in the present connection are to be understood as meaning non-living materials, such as, preferably, plastics, adhesives, sizes, papers and cards, leather, wood and processed wood products and coating compositions.

Wood and processed wood products are materials to be protected, especially preferably, from insect infestation.

Wood and processed wood products which can be protected by the agent according to the invention or mixtures comprising it are to be understood as meaning, for example:

building timber, wooden beams, railway sleepers, bridge components, boat jetties, wooden vehicles, boxes, pallets, containers, telegraph poles, wood panelling, wooden window frames and doors, plywood, chipboard, joinery or wooden products which are used quite generally in house-building or in building joinery.

The active compounds can be used as such, in the form of concentrates or in generally customary formulations, such as powders, granules, solutions, suspensions, emulsions or pastes.

The formulations mentioned can be prepared in a manner known per se, for example by mixing the active compounds with at least one solvent or diluent, emulsifier, dispersing agent and/or binder or fixing agent, a water repellent, if appropriate siccatives and UV stabilizers and if appropriate dyestuffs and pigments, and also other processing auxiliaries.

The insecticidal compositions or concentrates used for the protection of wood and wood-derived timber products comprise the active compound according to the invention in a concentration of 0.0001 to 95% by weight, in particular 0.001 to 60% by weight.

The amount of the compositions or concentrates employed depends on the nature and occurrence of the insects and on the medium. The optimum amount employed can be determined for the use in each case by series of tests. In general, however, it is sufficient to employ 0.0001 to 20% by weight, preferably 0.001 to 10% by weight, of the active compound, based on the material to be protected.

Solvents and/or diluents which are used are an organic chemical solvent or solvent mixture and/or an oily or oil-like organic chemical solvent or solvent mixture of low volatility and/or a polar organic chemical solvent or solvent mixture and/or water, and if appropriate an emulsifier and/or wetting agent.

Organic chemical solvents which are preferably used are oily or oil-like solvents having an evaporation number above 35 and a flashpoint above 30° C., preferably above 45° C. Substances which are used as such oily or oil-like water-insoluble solvents of low volatility are appropriate mineral oils or aromatic fractions thereof, or solvent mixtures containing mineral oils, preferably white spirit, petroleum and/or alkylbenzene.

Mineral oils having a boiling range from 170 to 220° C., white spirit having a boiling range from 170 to 220° C., spindle oil having a boiling range from 250 to 350° C., petroleum and aromatics having a boiling range from 160 to 280° C., terpentine oil and the like, are advantageously employed.

In a preferred embodiment, liquid aliphatic hydrocarbons having a boiling range from 0.180 to 210° C. or high-boiling mixtures of aromatic and aliphatic hydrocarbons having a boiling range from 180 to 220° C. and/or spindle oil and/or monochloronaphthalene, preferably α-monochloronaphthalene, are used.

The organic oily or oil-like solvents of low volatility which have an evaporation number above 35 and a flashpoint above 30° C., preferably above 45° C., can be replaced in part by organic chemical solvents of high or medium volatility, provided that the solvent mixture likewise has an evaporation number above 35 and a flashpoint above 30° C., preferably above 45° C., and that the insecticide/fungicide mixture is soluble or emulsifiable in this solvent mixture.

According to a preferred embodiment, some of the organic chemical solvent or solvent mixture is replaced by an aliphatic polar organic chemical solvent or solvent mixture. Aliphatic organic chemical solvents containing hydroxyl and/or ester and/or ether groups, such as, for example, glycol ethers, esters or the like, are preferably used.

Organic chemical binders which are used in the context of the present invention are the synthetic resins and/or binding drying oils which are known per se, are water-dilutable and/or are soluble or dispersible or emulsifiable in the organic chemical solvents employed, in particular binders consisting of or comprising an acrylate resin, a vinyl resin, for example polyvinyl acetate, polyester resin, polycondensation or polyaddition resin, polyurethane resin, alkyd resin or modified alkyd resin, phenolic resin, hydrocarbon resin, such as indene-coumarone resin, silicone resin, drying vegetable oils and/or drying oils and/or physically drying binders based on a natural and/or synthetic resin.

The synthetic resin used as the binder can be employed in the form of an emulsion, dispersion or solution. Bitumen or bituminous substances can also be used as binders in an amount of up to 10% by weight. Dyestuffs, pigments, water-repelling agents, odour correctants and inhibitors or anticorrosive agents and the like which are known per se can additionally be employed.

It is preferred according to the invention for the composition or concentrate to comprise, as the organic chemical binder, at least one alkyd resin or modified alkyd resin and/or a drying vegetable oil. Alkyd resins having an oil content of more than 45% by weight, preferably 50 to 68% by weight, are preferably used according to the invention.

All or some of the binder mentioned can be replaced by a fixing agent (mixture) or a plasticizer (mixture). These additives are intended to prevent evaporation of the active compounds and crystallization or precipitation. They preferably replace 0.01 to 30% of the binder (based on 100% of the binder employed).

The plasticizers originate from the chemical classes of phthalic acid esters, such as dibutyl, dioctyl or benzyl butyl phthalate, phosphoric acid esters, such as tributyl phosphate, adipic acid esters, such as di-(2-ethylhexyl) adipate, stearates, such as butyl stearate or amyl stearate, oleates, such as butyl oleate, glycerol ethers or higher molecular weight glycol ethers, glycerol esters and p-toluenesulphonic acid esters.

Fixing agents are based chemically on polyvinyl alkyl ethers, such as, for example, polyvinyl methyl ether or ketones, such as benzophenone or ethylenebenzophenone.

Possible solvents or diluents are, in particular, also water, if appropriate as a mixture with one or more of the abovementioned organic chemical solvents or diluents, emulsifiers and dispersing agents.

Particularly effective preservation of wood is achieved by impregnation processes on a large industrial scale, for example vacuum, double vacuum or pressure processes.

The ready-to-use compositions can also comprise other insecticides, if appropriate, and also one or more fungicides, if appropriate.

Possible additional mixing partners are, preferably, the insecticides and fungicides mentioned in WO 94/29 268. The compounds mentioned in this document are an explicit constituent of the present application.

Very particularly preferred mixing partners which may be mentioned are insecticides, such as chlorpyriphos, phoxim, silafluofin, alphamethrin, cyfluthrin, cypermethrin, deltamethrin, permethrin, imidacloprid, NI-25, flufenoxuron, hexaflumuron, transfluthrin, thiacloprid, methoxyfenozide and triflumuron, and also fungicides, such as epoxyconazole, hexaconazole, azaconazole, propiconazole, tebuconazole, cyproconazole, metconazole, imazalil, dichlorfluanid, tolylfluanid, 3-iodo-2-propinyl butylcarbamate, N-octyl-isothiazolin-3-one and 4,5-dichloro-N-octylisothiazolin-3-one.

The compounds according to the invention can at the same time be employed for protecting objects which come into contact with salt water or brackish water, in particular hulls, screens, nets, buildings, moorings and signalling systems, against fouling.

Fouling by sessile Oligochaeta, such as Serpulidae, and by shells and species from the Ledamoipha group (goose barnacles), such as various Lepas and Scalpellum species, or by species from the Balanomorpha group (acorn barnacles), such as Balanus or Pollicipes species, increases the frictional drag of ships and, as a consequence, leads to a marked increase in operation costs owing to higher energy consumption and additionally frequent residence in the dry dock.

Apart from fouling by algae, for example Ectocarpus sp. and Ceramium sp., fouling by sessile Entomostraka groups, which come under the generic term Cirripedia (cirriped crustaceans), is of particular importance.

Surprisingly, it has now been found that the compounds according to the invention, alone or in combination with other active compounds, have an outstanding antifouling action.

Using the compounds according to the invention, alone or in combination with other active compounds, allows the use of heavy metals such as, for example, in bis(trialkyltin) sulphides, tri-n-butyltin laurate, tri-n-butyltin chloride, copper(I) oxide, triethyltin chloride, tri-n-butyl(2-phenyl-4-chlorophenoxy)-tin, tributyltin oxide, molybdenum disulphide, antimony oxide, polymeric butyl titanate, phenyl-(bispyridine)-bismuth chloride, tri-n-butyltin fluoride, manganese ethylenebisthio-carbamate, zinc dimethyldithiocarbamate, zinc ethylenebisthiocarbamate, zinc salts and copper salts of 2-pyridinethiol 1-oxide, bisdimethyldithiocarbamoylzinc ethylene-bisthiocarbamate, zinc oxide, copper(I) ethylene-bisdithiocarbamate, copper thiocyanate, copper naphthenate and tributyltin halides to be dispensed with, or the concentration of these compounds to be substantially reduced.

If appropriate, the ready-to-use antifouling paints can additionally comprise other active compounds, preferably algicides, fungicides, herbicides, molluscicides, or other antifouling active compounds.

Preferably suitable components in combinations with the antifouling compositions according to the invention are:

algicides such as

2-tert-butylamino-4-cyclopropylamino-6-methylthio-1,3,5-triazine, dichlorophen, diuron, endothal, fentin acetate, isoproturon, methabenzthiazuron, oxyfluorfen, quinoclamine and terbutryn;

fungicides such as

benzo[b]thiophenecarboxylic acid cyclohexylamide S,S-dioxide, dichlofluanid, fluorfolpet, 3-iodo-2-propinyl butylcarbamate, tolylfluanid and azoles such as azaconazole, cyproconazole, epoxyconazole, hexaconazole, metconazole, propiconazole and tebuconazole;

molluscicides such as

fentin acetate, metaldehyde, methiocarb, niclosamid, thiodicarb and trimethacarb; or conventional antifouling active compounds such as 4,5-dichloro-2-octyl-4-isothiazolin-3-one, diiodomethylparatryl sulphone, 2-(N,N-dimethylthiocarbamoylthio)-5-nitrothiazyl, potassium, copper, sodium and zinc salts of 2-pyridinethiol 1-oxide, pyridine-triphenylborane, tetrabutyldistannoxane, 2,3,5,6-tetrachloro-4-(methylsulphonyl)-pyridine, 2,4,5,6-tetrachloroisophthalonitrile, tetramethylthiuram disulphide and 2,4,6-trichlorophenylmalei mide.

The antifouling compositions used comprise the active compound according to the invention of the compounds according to the invention in a concentration of 0.001 to 50% by weight, in particular 0.01 to 20% by weight.

Moreover, the antifouling compositions according to the invention comprise the customary components such as, for example, those described in Ungerer, Chem. Ind. 1985, 37, 730-732 and Williams, Antifouling Marine Coatings, Noyes, Park Ridge, 1973.

Besides the algicidal, fungicidal, molluscicidal active compounds and insecticidal active compounds according to the invention, antifouling paints comprise, in particular, binders.

Examples of recognized binders are polyvinyl chloride in a solvent system, chlorinated rubber in a solvent system, acrylic resins in a solvent system, in particular in an aqueous system, vinyl chloride/vinyl acetate copolymer systems in the form of aqueous dispersions or in the form of organic solvent systems, butadiene/styrene/acrylonitrile rubbers, drying oils such as linseed oil, resin esters or modified hardened resins in combination with tar or bitumens, asphalt and epoxy compounds, small amounts of chlorine rubber, chlorinated polypropylene and vinyl resins.

If appropriate, paints also comprise inorganic pigments, organic pigments or colorants which are preferably insoluble in salt water. Paints may furthermore comprise materials such as colophonium to allow controlled release of the active compounds. Furthermore, the paints may comprise plasticizers, modifiers which affect the rheological properties and other conventional constituents. The compounds according to the invention or the abovementioned mixtures may also be incorporated into self-polishing antifouling systems.

The active compounds are also suitable for controlling animal pests, in particular insects, arachnids and mites, which are found in enclosed spaces such as, for example, dwellings, factory halls, offices, vehicle cabins and the like. They can be employed alone or in combination with other active compounds and auxiliaries in domestic insecticide products for controlling these pests. They are active against sensitive and resistant species and against all development stages. These pests include:

From the order of the Scorpionidea ,for example, Buthus occitanus.

From the order of the Acarina ,for example, Argas persicus, Argas reflexus, Bryobia spp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus and Dermatophagoides forinae.

From the order of the Araneae ,for example, Aviculariidae and Araneidae.

From the order of the Opiliones ,for example, Pseudoscorpiones chelifer, Pseudoscorpiones cheiridium and Opiliones phalangium.

From the order of the Isopoda, for example, Oniscus asellus and Porcellio scaber.

From the order of the Diplopoda ,for example, Blaniulus guttulatus and Polydesmus spp.

From the order of the Chilopoda, for example, Geophilus spp.

From the order of the Zygentoma ,for example, Ctenolepisma spp., Lepisma saccharina and Lepismodes inquilinus.

From the order of the Blattaria ,for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa and Supella longipalpa.

From the order of the Saltatoria, for example, Acheta domesticus.

From the order of the Dermaptera ,for example, Forficula auricularia.

From the order of the Isoptera ,for example, Kalotermes spp. and Reticulitermes spp.

From the order of the Psocoptera, for example, Lepinatus spp. and Liposcelis spp.

From the order of the Coleptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais and Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga camaria, Simulium spp., Stomoxys calcitrans and Tipula paludosa.

From the order of the Lepidoptera, for example, Achroia grisella, Galleria mellonella, Plodia interpunctella, Tinea cloacella, Tinea pellionella and Tineola bisselliella.

From the order of the Siphonaptera, for example, Ctenocephalides canis, Ctenocephalides felis, Pulex irritans, Tunga penetrans and Xenopsylla cheopis.

From the order of the Hymenoptera, for example, Camponotus herculeanus, Lasius fuliginosus, Lasius niger, Lasius umbratus, Monomorium pharaonis, Paravespula spp. and Tetramorium caespitum.

From the order of the Anoplura, for example, Pediculus humanus capitis, Pediculus humanus corporis and Phthirus pubis.

From the order of the Heteroptera, for example, Cimex hemipterus, Cimex lectularius, Rhodinus prolixus and Triatoma infestans.

In the field of household insecticides, they are used alone or in combination with other suitable active compounds, such as phosphoric acid esters, carbamates, pyrethroids, growth regulators or active compounds from other known classes of insecticides.

They are used as aerosols, unpressurized spray products, for example pump and atomizer sprays, automatic misting systems, foggers, foams, gels, evaporator products with evaporator tablets made of cellulose or polymer, liquid evaporators, gel and membrane evaporators, propeller-driven evaporators, unpowered or passive evaporation systems, moth papers, moth bags and moth gels, as granules or dusts, in baits for spreading or in bait stations.

### PREPARATION EXAMPLES

### Example I-1

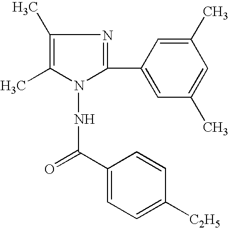

A solution of p-ethylbenzoyl chloride (1.685 g, 10 mM) in 10 ml of absolute tetrahydrofuran (THF) is added under argon at 0° C. to the compound from Preparation Example II-1 (1.075 g, 5 mM) and Hüinig's base (2.61 ml, 15 mM) in 10 ml of abs. THF and the mixture is stirred at 0° C. for 15 minutes and then at room temperature overnight. The reaction solution is poured into pH 7 buffer and extracted 3× with ethyl acetate. The organic phase is dried and concentrated. The residue is dissolved in 20 ml of methanol, 6 ml of concentrated sodium hydroxide solution are added and the mixture is stirred at room temperature for 1 hour. It is poured into water and the pH is adjusted to 7 using concentrated hydrochloric acid. It is extracted 3× with ethyl acetate. The organic phase is dried and concentrated.

Yield: 1.3 g (75% of theory), m.p. 234° C., logP (pH 2.3) 1.97.

### Example I-2

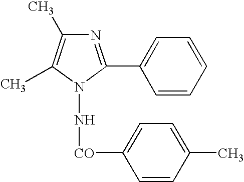

A mixture of benzaldehyde (3.18 g, 0.03 M), p-tolylic hydrazide (4.5 g, 0.03 M) and 2,3-butanedione monoxime (9.09 g, 0.09 M) in 90 ml of glacial acetic acid is heated under reflux overnight and then concentrated. The residue is taken up in dichloromethane/water, sodium carbonate solution is added and the organic phase is separated. The organic phase is washed with water, dried and concentrated. The crude product (10.7 g) is extracted by stirring with ether and the solid (5.8 g) is filtered off with suction. This solid (N-oxide) is washed with water, filtered off with suction, extracted by stirring with ethyl acetate/ethanol 3:1 and filtered off with suction again.

Yield (N-oxide): 3.7 g (38.4% of theory), m.p.:>250° C., logP (pH 2.3) 1.51.

Sodium iodide (1.75 g, 0.0117 M) and, immediately thereafter, a solution of the above N-oxide (0.63 g, 0.00196 M) in 3 ml of absolute acetonitrile are added to a solution of titanium tetrachloride (0.74 g, 0.0039 M) in 10 ml of absolute acetonitrile.

The mixture is subsequently stirred at room temperature for 40 minutes, water is added and the pH is adjusted to 7 using dilute potassium hydroxide solution, the mixture is filtered and the filtrate is extracted with dichloromethane. The target product is recovered from the dichloromethane extract.

Yield: 0.23 g, m.p.: 280° C., logP (pH 7.5) 2.17.

In analogy to the examples and in accordance with the general preparation details, the compounds of the formula (I) indicated in Table I below are obtained:

TABLE 1

| | |
|---|---|
| | (I) |
| 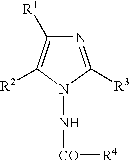 | |

| Ex. | | | | | logP |
|---|---|---|---|---|---|
| No. | R¹ | R² | R³ | R⁴ | (pH 2.3) |
| 3 | CH₃ | CH₃ | 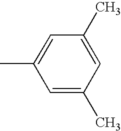 | 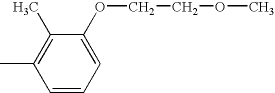 | 1.68 |
| 4 | CH₃ | CH₃ | 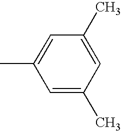 | 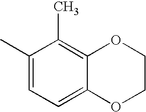 | 1.68 |
| 5 | CH₃ | CH₃ | 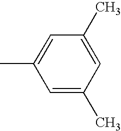 | 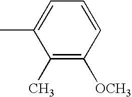 | 1.74 |
| 6 | H | CH₃ | 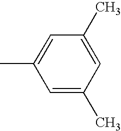 | 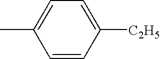 | 1.90 |
| 7 | H | CH₃ | 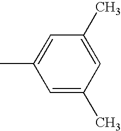 | 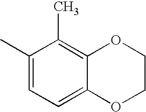 | 1.64 |
| 8 | H | H | 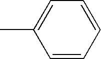 | 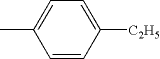 | 1.51 |
| 9 | 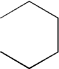 | | 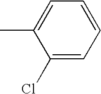 | 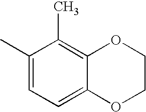 | 2.42 (pH 7.5) |
| 10 | CH₃ | CH₃ | 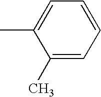 | 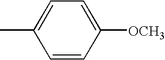 | 2.06 (pH 7.5) |
| 11 | CH₃ | CH₃ | 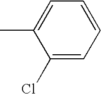 | 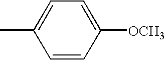 | 2.05 (pH 7.5) |
| 12 | CH₃ | CH₃ | 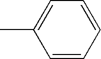 | 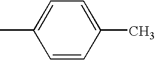 | 1.44 |
| 13 | CH₃ | CH₃ | 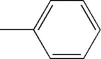 | 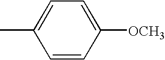 | 1.32 |
| 14 | CH₃ | CH₃ | 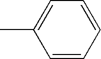 | 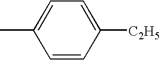 | 1.65 |
| 15 | CH₃ | CH₃ | 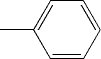 | 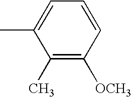 | 1.44 |
| 16 | CH₃ | 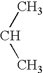 | 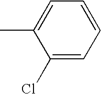 | 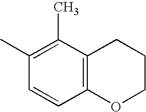 | 1.96 |
| 17 | CH₃ | CH₃ | 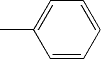 | 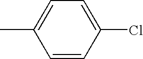 | 1.53 |
| 18 | CH₃ | CH₃ | 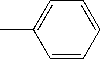 | 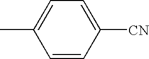 | 1.23 |
| 19 | CH₃ | CH₃ | 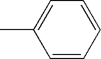 | 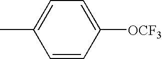 | 1.77 |
| 20 | H | CH₃ | 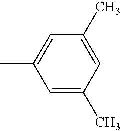 | 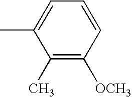 | 1.67 |
| 21 | H | H | 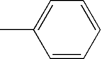 | 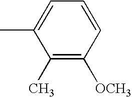 | 1.29 |
| 22 | H | CH₃ | 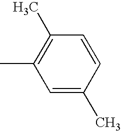 | 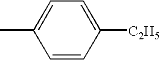 | 1.84 |
| 23 | H | CH₃ | 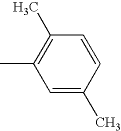 | 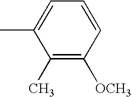 | 1.62 |
| 24 | H | H | 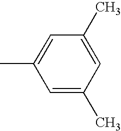 | 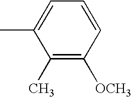 | 1.60 |
| 25 | H | CH₃ | 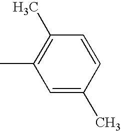 | 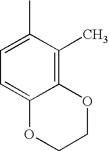 | 1.54 |
| 26 | H | CH₃ | 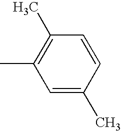 | 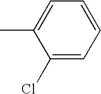 | 1.48 |
| 27 | H | CH₃ | 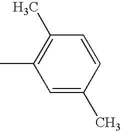 | 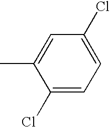 | 1.75 |
| 28 | H | CH₃ | 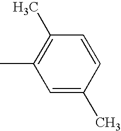 | 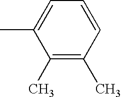 | 1.67 |
| 29 | H | CH₃ | 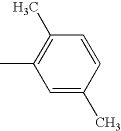 | 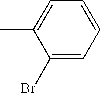 | 1.52 |
| 30 | H | CH₃ | 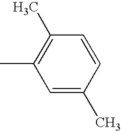 | 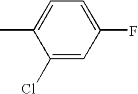 | 1.60 |
| 31 | H | CH₃ | 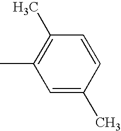 | 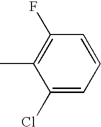 | 1.50 |
| 32 | H | CH₃ | 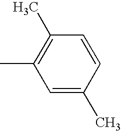 | 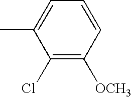 | 1.48 |
| 33 | H | CH₃ | 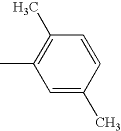 | 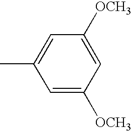 | 1.61 |
| 34 | H | CH₃ | 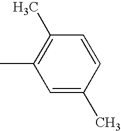 | 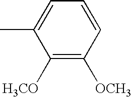 | 1.61 |
| 35 | H | CH₃ | 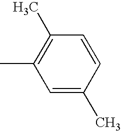 | 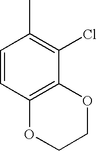 | 1.47 |
| 36 | H | CH₃ | 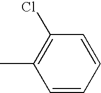 | 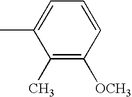 | 1.47 |
| 37 | H | CH₃ | 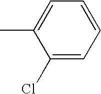 | 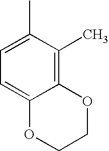 | 1.41 |
| 38 | H | CH₃ | 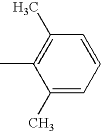 | 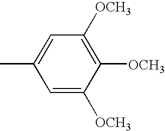 | 1.52 |
| 39 | H | CH₃ | 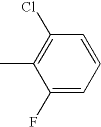 | 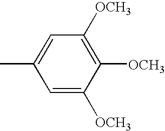 | 1.34 |
| 40 | H | CH₃ | 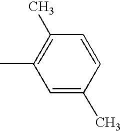 | 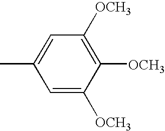 | 1.54 |
| 41 | H | CH₃ | 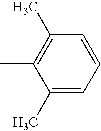 | 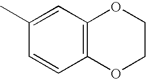 | 1.46 |
| 42 | H | CH₃ | 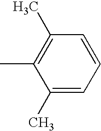 | 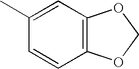 | 1.42 |
| 43 | H | CH₃ | 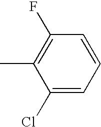 | 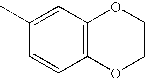 | 1.27 |
| 44 | H | CH₃ | 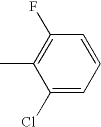 | 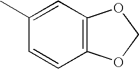 | 1.24 |
| 45 | H | CH₃ | 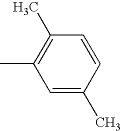 | 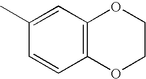 | 1.49 |
| 46 | H | CH₃ | 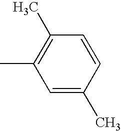 | 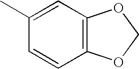 | 1.47 |
| 47 | H | CH₃ | 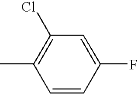 | 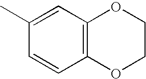 | 1.39 |
| 48 | H | CH₃ | 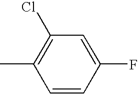 | 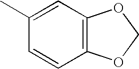 | 1.35 |
| 49 | H | CH₃ | 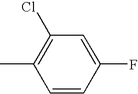 | 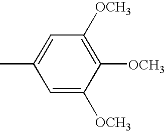 | 1.47 |
| 50 | H | CH₃ | 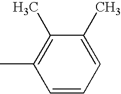 | 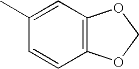 | 1.45 |
| 51 | H | CH₃ | 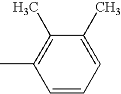 | 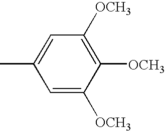 | 1.51 |
| 52 | H | CH₃ | 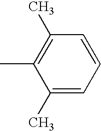 | 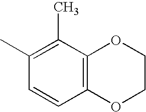 | 1.56 |
| 53 | H | CH₃ | 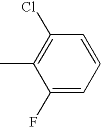 | 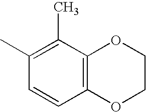 | 1.40 |
| 54 | H | CH₃ | 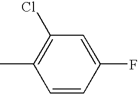 | 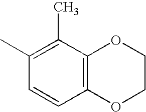 | 1.48 |
| 55 | H | CH₃ | 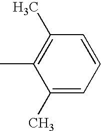 | 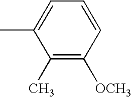 | 1.62 |
| 56 | H | CH₃ | 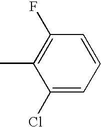 | 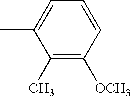 | 1.49 |
| 57 | H | CH₃ | 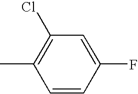 | 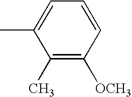 | 1.56 |
| 58 | H | CH₃ | 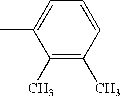 | 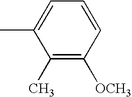 | 1.68 |
| 59 | H | CH₃ | 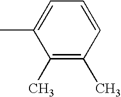 | 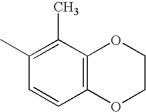 | 1.61 |
| 60 | H | CH₃ | 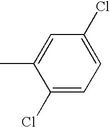 | 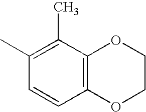 | 1.66 |
| 61 | H | CH₃ | 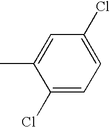 | 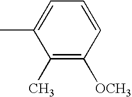 | 1.76 |
| 62 | H | CH₃ | 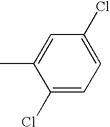 | 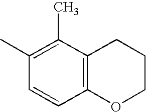 | 1.88 |
| 63 | H | CH₃ | 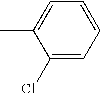 | 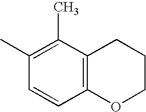 | 1.59 |
| 64 | H | CH₃ | 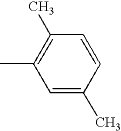 | 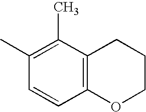 | 1.80 |
| 65 | CH₃ | CH₃ | 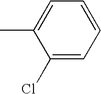 | 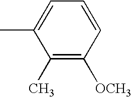 | 1.53 |
| 66 | CH₃ | CH₃ | 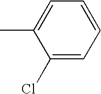 | 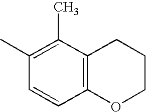 | 1.68 |
| 67 | CH₃ | CH₃ | 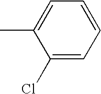 | 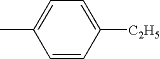 | 1.85 |
| 68 | H | CH₃ | 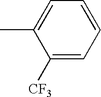 | 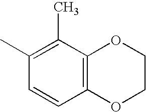 | 2.21 (pH 7.5) |
| 69 | H | CH₃ | 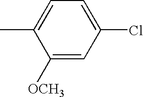 | 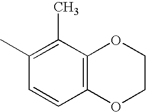 | 1.61 |
| 70 | H | CH₃ | 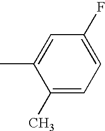 | 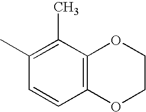 | 2.17 (pH 7.5) |
| 71 | H | CH₃ | 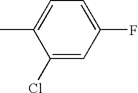 | 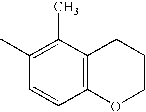 | 2.35 (pH 7.5) |
| 72 | H | CH₃ | 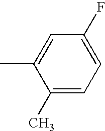 | 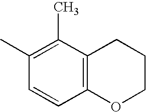 | 2.40 (pH 7.5) |
| 73 | H | CH₃ | 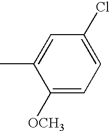 | 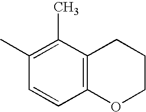 | 2.44 (pH 7.5) |
| 74 | H | CH₃ | 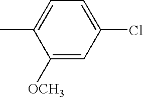 | 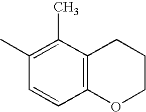 | 2.46 (pH 7.5) |
| 75 | CH₃ | CH₃ | 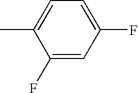 | 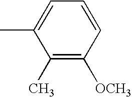 | 2.24 (pH 7.5) |
| 76 | CH₃ | CH₃ | 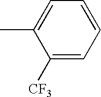 | 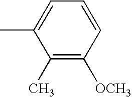 | 2.25 (pH 7.5) |
| 77 | CH₃ | 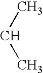 | 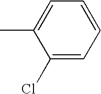 | 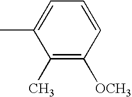 | 1.79 |
| 78 | CH₃ | 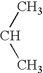 | 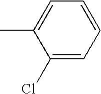 | 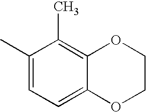 | 1.73 |
| 79 | CH₃ | 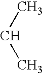 | 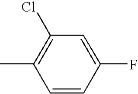 | 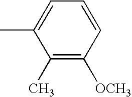 | 1.94 |
| 80 | CH₃ | CH₃ | 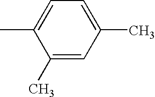 | 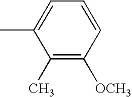 | 2.48 (pH 7.5) |
| 81 | CH₃ | 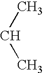 | 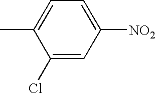 | 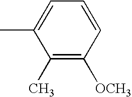 | 2.18 |
| 82 | CH₃ | CH₃ | 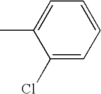 | 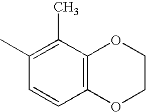 | 1.53 |
| 83 | CH₃ | CH₃ | 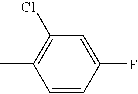 | 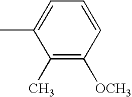 | 2.35 (pH 7.5) |
| 84 | CH₃ | 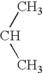 | 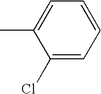 | 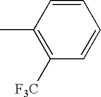 | 1.88 |
| 85 | CH₃ | CH₃ | 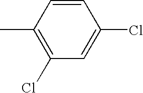 | 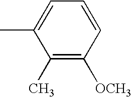 | 2.61 (pH 7.5) |
| 86 | CH₃ | CH₃ | 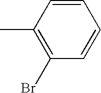 | 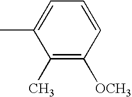 | 2.25 (pH 7.5) |
| 87 | CH₃ | 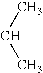 | 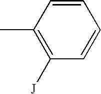 | 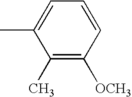 | 1.93 |
| 88 | CH₃ | CH₃ | 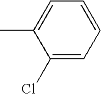 | 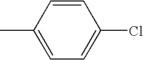 | 2.38 (pH 7.5) |
| 89 | CH₃ | CH₃ | 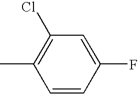 | 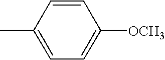 | 1.52 |
| 90 | CH₃ | CH₃ | 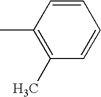 | 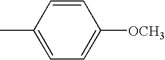 | 2.06 (pH 7.5) |
| 91 | CH₃ | 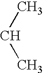 | 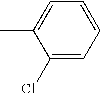 | 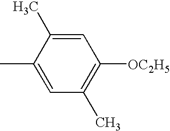 | 2.46 |
| 92 | CH₃ | CH₃ | 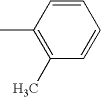 | 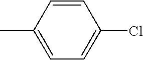 | 2.39 (pH 7.5) |
| 93 | CH₃ | CH₃ | 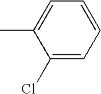 | 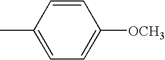 | 1.41 |
| 94 | CH₃ | 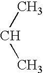 | 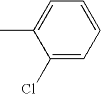 | 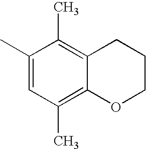 | 2.28 |
| 95 | CH₃ | 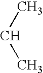 | 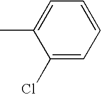 | 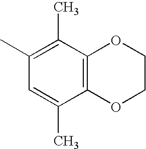 | 1.97 |
| 96 | CH₃ | CH₃ | 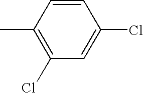 | 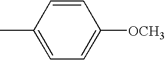 | 1.72 |

| | | | | |
|---|---|---|---|---|
| 97 | 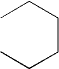 | 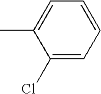 | 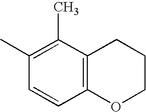 | 1.87 |
| 98 | 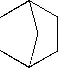 | 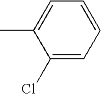 | 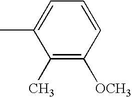 | |

### Example II-1

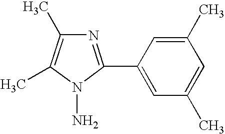

Concentrated H₂SO₄ (100 ml) is added to the compound from Preparation Example IV-1 (10 g, 0.0271 M) and the mixture is stirred at RT for 3 h, poured very slowly onto a large quantity of ice, rendered alkaline with 10% NaOH and extracted 3× with ethyl acetate.

Yield: 4.73 g (81% of theory), logP (pH 2.3) 0.96.

### Example III-1

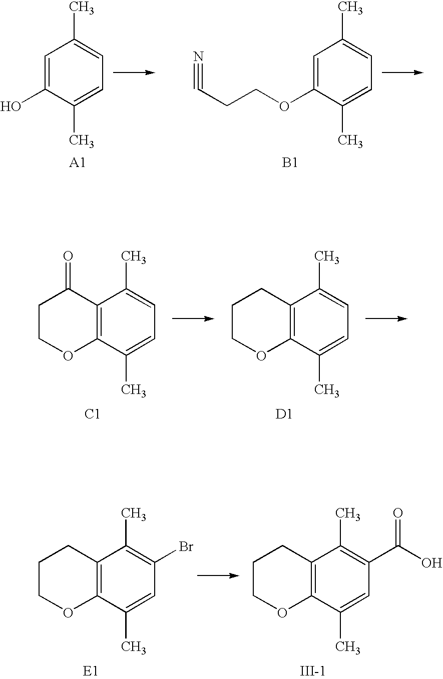

Benzyltrimethylammonium hydroxide (40% strength solution in methanol, 10 g; 0.06M) is added slowly dropwise at −10° C. to a mixture of 2,5-dimethylphenol (60 g; 0.491M) and acrylonitrile (156.9 g; 2.957M). The mixture is slowly brought to room temperature, then boiled under reflux for 3 days and concentrated. The residue is washed with dilute sodium chloride solution and extracted with chloroform.

This gives 105.93 g of the crude product B1 (purity 71% by GC/MS, remainder starting phenol).

The crude product B1 (50 g, 0.2026M) is introduced dropwise at 180-185° C. into rapidly stirred polyphosphoric acid (84% strength, 1 028 g) and the mixture is held at this temperature for 2.5 h. The hot reaction mixture is poured into ice-water and extracted with chloroform.

This gives 22.9 g of the crude product C1 (purity 89% by LC/MS).

The crude product C1 (22.9 g, 0.1157M), dissolved in 130 ml of trifluoroacetic acid, is admixed dropwise at room temperature with triethylsilane (66.86 g; 0.575M) and the mixture is stirred for 33 h, poured onto ice and extracted with chloroform. The crude product is purified by chromatography (silica gel, cyclohexane).

This gives 11.13 g of the compound D1 (purity 85% by GC/MS).

The compound D1 (16.0 g, 0.084M), dissolved in 150 ml of anhydrous chloroform, is admixed at 0° C. with a spatula tip of aluminium chloride and then with bromine (4.32 ml; 0.0839M); the temperature is held below 4° C. during the addition. The mixture is subsequently stirred at room temperature for 1 h, water is added, and the mixture is extracted with chloroform.

Purification of the extract by column chromatography (silica gel, cyclohexane) gives 18.3 g of the compound El (purity 95% by GC/MS).

The compound El (18.0 g, 0.071M) is dissolved in 200 ml of anhydrous THF, the solution is cooled to −70° C. under argon, n-butyllithium (15% strength in hexane, 46.22 ml; 0.075M) is added dropwise, and the mixture is stirred at −70° C. for 1 h and then poured onto dry ice. After 8 h, water is added, acidification is carried out with dilute hydrochloric acid and the solid is filtered off with suction.

This gives 11.44 g of the compound III-1 (purity 98% by LC/MS), m.p. 211.5° C.

### Example III-2

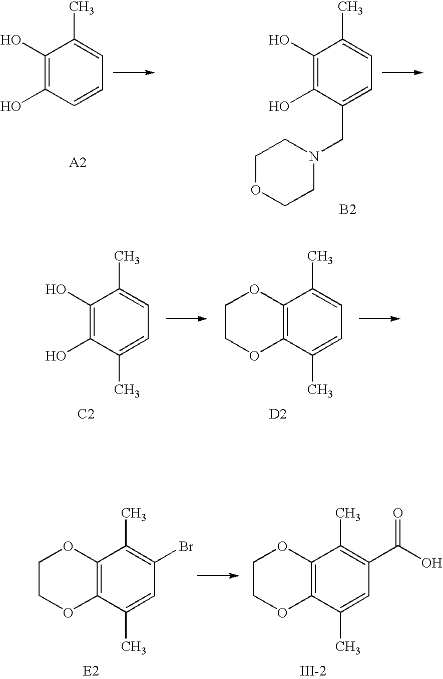

Morpholine (86 g; 0.987M) and formaldehyde (35% strength solution in water, 86 g; IM) in 250 ml of isopropanol are boiled under reflux for 10 minutes and cooled to 0° C. and a solution of 3-methylpyrocatechol (124 g; IM) in 400 ml of isopropanol is added dropwise over the course of 15 minutes. The reaction mixture is boiled under reflux for 15 minutes, cooled and concentrated in vacuo and the residue is recrystallized from isopropanol.

This gives 156.5 g of the compound B2 (purity 99% by LC/MS).

The compound B2 (117.44 g; 0.526M) is dissolved in 2 1 of ethanol, 48 g of 10% palladium on charcoal are added and the mixture is hydrogenated at 80° C. overnight under 60 bar of hydrogen. The hydrogenation mixture is freed from the catalyst by filtration over Kieselguhr, the filtrate is concentrated in vacuo, the residue is dissolved in chloroform and the solution is washed in succession with dilute hydrochloric acid and saturated sodium chloride solution, dried and concentrated in vacuo.

This gives 34.02 g of the compound C2 (purity 85% by LC/MS).

A suspension of the compound C2 (36.061 g, 0.222M) and dried, powdered potassium carbonate (108.2 g; 0.783M) in 250 ml of abs. DMF is heated to 110° C. under argon and dibromoethane (147.1 g; 0.783M) is added dropwise over 2 h. After the end of the addition, heating is continued at 110° C. for 30 minutes, the mixture is cooled and filtered with suction, the filtrate is concentrated in vacuo and the residue is dissolved in chloroform and washed 2× with dilute sodium hydroxide solution and 1× with saturated sodium chloride solution.

This gives 35.7 g of the compound D2 (purity 83% by LC/MS).

N-Bromosuccinimide (22.521 g; 0.1265M) is added at −30° C. to the compound D2 (22.891 g; 0.116M) in solution in 450 ml of anhydrous acetonitrile, cooling is removed and the batch is stirred overnight, filtered and concentrated in vacuo. The crude product is purified twice by column chromatography (silica gel, cyclohexane).

This gives 11.6 g of the compound E2 (purity 88% by GC/MS).

n-Butyllithium (15% strength in hexane, 29.58 ml; 0.048M) is added dropwise at −70° C. to the compound E2 (11.6 g, 0.042M) in 100 ml of anhydrous THF and the mixture is stirred at −70° C. for 1 h, then poured onto dry ice and left to stand overnight. Dilute sodium hydroxide solution is added to the residue, the mixture is washed 2× with ether, and acidified with concentrated hydrochloric acid, and the solid is filtered off with suction and dried.

This gives 6.56 g of the compound III-2 (purity 86% by BPLC, contaminated with 12% of 5,7,8-trimethyl-1,4-benzodioxane-6-carboxylic acid), m.p. 202-3° C.

### Example III-3

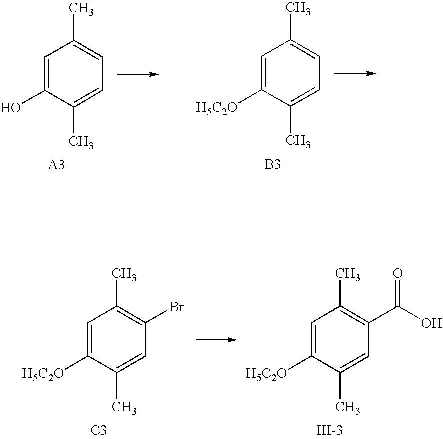

Iodoethane (78 g; 0.5M) is added to a suspension of 2,5-dimethylphenol (40 g; 0.327M) and dried, powdered potassium carbonate (45.8 g; 0.331M) in 70 ml of anhydrous acetone and the mixture is heated and stirred under reflux overnight. After cooling, water is added to the reaction mixture and extraction is carried out 2× with dichloromethane. The combined organic phases are washed 2× with 10% strength sodium hydroxide solution, 1× with saturated sodium chloride solution, dried and concentrated in vacuo.

This gives 40.25 g of the compound B3 (purity 99% by HPLC).

A solution of the compound B3 (40.25 g; 0.268M) in 450 ml of anhydrous chloroform is cooled to 0° C., and a spatula tip of aluminium chloride followed dropwise by bromine (16.8 ml; 0.326M) are added. The mixture is stirred at 0° C. for 1 h, then stirred at room temperature for 1 h, water is added and the chloroform phase is separated off. Washing is carried out with sodium bicarbonate solution and sodium chloride solution and the washed mixture is dried and concentrated in vacuo. It is then purified by column chromatography (silica gel, cyclohexane).

This gives 29.22 g of the compound C3 (purity 99% by BPLC).

A solution of the compound C3 (28.2 g; 0.123M) in 250 ml of anhydrous THF is cooled to −60° C. under argon, n-butyllithium (15% strength in hexane, 90 ml; 0.146M) is added dropwise, and the mixture is then stirred at −60° C. for 1.5 h and subsequently poured onto dry ice. It is left to stand overnight, water is added to the residue, the mixture is acidified with concentrated hydrochloric acid and the solid is filtered off with suction.

This gives 18.05 g of the compound III-3 (purity 98% by HPLC), m.p. 178° C.

### Example IV-1

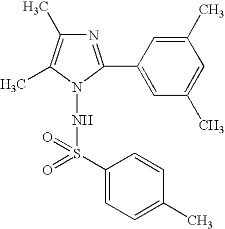

The compound from Preparation Example V-1 (32.3 g, 0.08346 M) and POCl₃ (15.5 ml, 0.16692 M) are stirred under reflux in 650 ml of absolute chloroform for 3 hours. A further 15.5 ml of POCl₃ are added and the mixture is stirred under reflux overnight. With ice cooling, 450 ml of water are carefully added to the reaction solution which is stirred for 2 hours before 3 1 of saturated NaHCO₃ solution are slowly added. The organic phase is separated off and the aqueous phase is extracted 2× with chloroform. The organic phases are dried and concentrated. The product is purified by column chromatography on silica gel (cyclohexane/ethyl acetate 1:2+a little CHCl₃).

Yield: 27.18 g (88% of theory), logP (pH 2.3) 1.69.

In analogy to the examples and in accordance with the general preparation details, the compounds of the formula (IV) indicated in Table 2 below are obtained:

TABLE 2

| | |
|---|---|
| | (IV) |
| 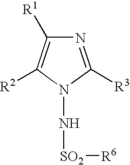 | |

| | | | | | logP |
|---|---|---|---|---|---|
| Ex. No. | R¹ | R² | R³ | R⁶ | (pH 2.3) |
| 2 | H | CH₃ | 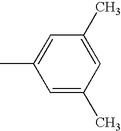 | 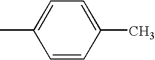 | 1.63 |
| 3 | CH₃ | CH₃ | 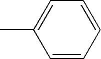 | 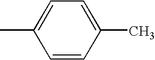 | 1.43 |
| 4 | H | CH₃ | 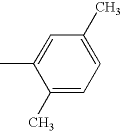 | 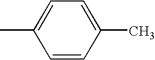 | 1.57 |
| 5 | H | CH₃ | 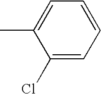 | 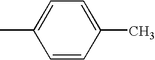 | 1.48 |
| 6 | H | CH₃ | 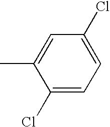 | 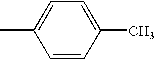 | 1.92 |
| 7 | H | CH₃ | 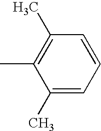 | 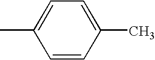 | 1.59 |
| 8 | H | CH₃ | 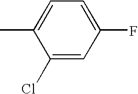 | 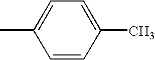 | 1.62 |
| 9 | H | CH₃ | 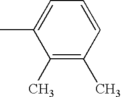 | 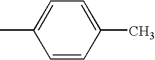 | 1.59 |
| 10 | H | CH₃ | 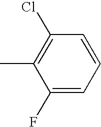 | 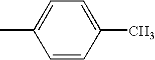 | 1.65 |
| 11 | H | CH₃ | 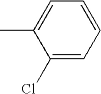 | 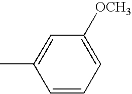 | 1.39 |
| 12 | CH₃ | CH₃ | 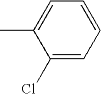 | 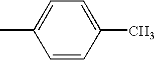 | 1.56 |
| 13 | H | CH₃ | 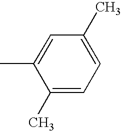 | 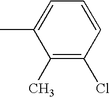 | 1.93 |
| 14 | H | CH₃ | 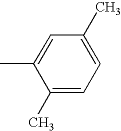 | 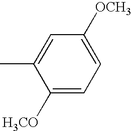 | |
| 15 | H | CH₃ | 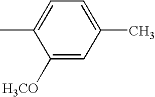 | 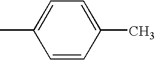 | 1.59 |
| 16 | H | CH₃ | 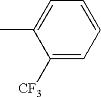 | 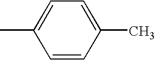 | 1.76 |
| 17 | H | CH₃ | 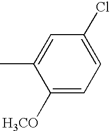 | 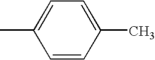 | 1.69 |
| 18 | H | CH₃ | 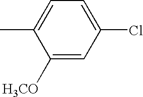 | 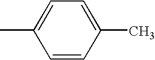 | 1.68 |
| 19 | H | CH₃ | 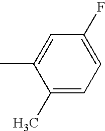 | 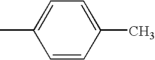 | 1.56 |
| 20 | CH₃ | 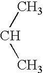 | 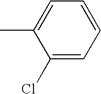 | 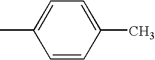 | 1.81 (pH 7.5) |

### Example V-1

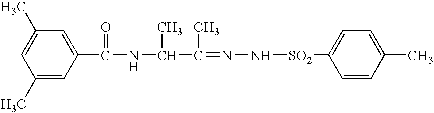

3,5-Dimethylbenzoyl chloride (42.1 g, 0.25 M) is added dropwise over the course of 2 hours at 10-15° C. to a solution of DL-alanine (44.55 g, 0.5 M) in 450 ml of 10% strength aqueous sodium hydroxide solution. The solution is neutralized with concentrated hydrochloric acid. The precipitate is filtered off with suction, washed with water and dried azeotropically in toluene on a water separator.

Yield (compound A): 29.5 g (53% of theory).

5 g of the compound A, DMAP (0.11 g, 0.9 mM), acetic anhydride (4.52 ml, 0.04746 M) and triethylamine (4.52 ml, 0.0327 M) are stirred at room temperature.

After 30 minutes 34 ml of glacial acetic acid are added to the reaction solution which is then left to stand for 30 minutes. The solvent is distilled off, the precipitate is taken up in water, the mixture is rendered alkaline using 2N sodium hydroxide and extracted with ether. The organic phase is washed with 1N hydrochloric acid solution and dried. The product is purified by column chromatography on silica gel (cyclohexane/ethyl acetate, 3/1).

Yield (compound B): 3.73 g (75% of theory), logP (pH 2.3) 1.93. 17 g of the compound B from the above batch, p-toluenesulfonyl hydrazide (14.38 g, 0.0776 M) and a spatula tip of PTSS are boiled in 1.81 of benzene on a water separator for 4 h and stirred at 0° C. for 15 minutes. The precipitate is filtered off with suction, washed with cold benzene and dried.

Yield: 27.3 g (91% of theory).

The crude product is reacted without further purification.

The logP values indicated were determined in accordance with EEC Directive 79/831 Annex V.A8 by means of HPLC (High Performance Liquid Chromatography) on a reversed-phase column (C 18). Temperature: 43° C.

(a) Eluents for determination in the acidic range: 0.1% aqueous phosphoric acid, acetonitriie; linear gradient from 10% acetonitrile to 90% acetonitrile.

(b) Eluents for determination in the neutral range: 0.01 molar aqueous phosphate buffer solution, acetonitrile; linear gradient from 10% acetonitrile to 90% acetonitrile.

Calibration was carried out with unbranched alkan-2-ones (having 3 to 16 carbon atoms) of known logP (logP values are determined on the basis of the retention times by means of linear interpolation between two successive alkanones).

The lambda max values were determined from the UV spectra from 200 nm to 400 nm in the maxima of the chromatographic signals.

### USE EXAMPLES

### Example A

Meloidogyne test

| | |
|---|---|
| Solvent: | 30 parts by weight of dimethylformamide |
| Emulsifier: | 1 part by weight of alkylaryl polyglycol ether |

To produce an appropriate preparation of active compound, 1 part by weight of active compound is mixed with the stated amounts of solvent and emulsifier and then the concentrate is diluted with water to the desired concentration.

Pots are filled with sand, active compound solution, Meloidogyne incognito egg/larvae suspension and lettuce seeds. The lettuce seeds germinate and the plantlets develop. The galls develop on the roots.

After the desired period of time, the nematicidal activity is determined in % from the formation of galls. 100% means that no galls were found; 0% denotes that the number of galls on the treated plants corresponds to the untreated control.

In this test, at an exemplary active compound concentration of 20 ppm, the compound from Preparation Example No. 1-52 exhibits a kill of 100% after 14 days.

### Example B

Phaedon larvae test

| | |
|---|---|
| Solvent: | 30 parts by weight of dimethylformamide |
| Emulsifier: | 1 part by weight of alkylaryl polyglycol ether |

To produce an appropriate preparation of active compound, 1 part by weight of active compound is mixed with the stated amounts of solvent and emulsifier and then the concentrate is diluted with emulsifier-containing water to the desired concentration.

Cabbage leaves (Brassica oleracea) are treated by being dipped into the preparation of active compound at the desired concentration and are populated with larvae of the mustard beetle (Phaedon cochleariae) while the leaves are still wet.

After the desired period of time, the kill in % is determined. 100% means that all the beetle larvae were killed, 0% means that no beetle larvae were killed.

In this test, at an exemplary active compound concentration of 1 000 ppm, the compound from Preparation Example No. I-82 exhibits a kill of 100% after 7 days.

### Example C

Spodoptera frugiperda test

| | |
|---|---|
| Solvent: | 30 parts by weight of dimethylformamide |
| Emulsifier: | 1 part by weight of alkylaryl polyglycol ether |

To produce an appropriate preparation of active compound, 1 part by weight of active compound is mixed with the stated amounts of solvent and emulsifier and then the concentrate is diluted with emulsifier-containing water to the desired concentration.

Cabbage leaves (Brassica oleracea) are treated by being dipped into the preparation of active compound at the desired concentration and are populated with caterpillars of the army worm (Spodoptera frugiperda) while the leaves are still wet.

After the desired period of time, the kill in % is determined. 100% means that all the caterpillars were killed, 0% means that no caterpillars were killed.

In this test, at an exemplary active compound concentration of 1 000 ppm, the compounds from Preparation Examples Nos. I-48, I-54, I-60, I-61, I-63, I-65, I-66, I-69, I-82 exhibit a kill of 100% after 7 days.

### Example D

Tetranychus test (OP resistant/dip treatment)

| | |
|---|---|
| Solvent: | 30 parts by weight of dimethylformamide |
| Emulsifier: | 1 part by weight of alkylaryl polyglycol ether |

To produce an appropriate preparation of active compound, 1 part by weight of active compound is mixed with the stated amounts of solvent and emulsifier and then the concentrate is diluted with emulsifier-containing water to the desired concentration.

Bean plants (Phaseolus vulgaris) heavily infested by all stages of the common spider mite (Tetranychus urticae) are dipped into a preparation of the active compound at the desired concentration.

After the desired period of time, the effect in % is determined. 100% means that all the spider mites were killed, 0% means that no spider mites were killed.

In this test, at an exemplary active compound concentration of 100 ppm, the compound from Preparation Example No. 1-4 exhibits a kill of 100% after 7 days.

### Example E

Aedes test

| | |
|---|---|
| Solvent: | 1 000 parts by weight of methanol |

To produce an appropriate preparation of active compound, 1 part by weight of active compound is mixed with the stated amount of solvent and the concentrate is diluted with a 0.01% strength lecithin solution to the desired concentrations.

Midge larvae (Aedes aegypti) are placed into the preparation of active compound at the desired concentration.

After the desired period of time, the effect on the larvae is determined. 100% means that all of the midges showed severe symptoms or have been killed, 0% means that no midges were harmed.

In this test, at an exemplary active compound concentration of 17 ppm, the compound from Preparation Example No. I-19 exhibits a kill of 100% after 4 hours.

### Example F

Blowfly larvae test/development inhibition activity

| | |
|---|---|
| Test organisms: | Lucilia cuprina larvae |
| Solvent: | Dimethyl sulphoxide |

20 mg of active compound are dissolved in 1 ml of dimethyl sulphoxide; lower concentrations are prepared by dilution with distilled H₂O.

About 20 Lucilia cuprina larvae are placed in a test tube containing about 1 cm³ of horse meat and 0.5 ml of the test active compound preparation. After 24 and 48 hours the efficacy of the active compound preparation is determined. The test tubes are transferred to beakers with sand-covered bases. After a further 2 days the test tubes are removed and the pupae are counted.

The effect of the active compound preparation is assessed according to the number of hatched flies after one and a half times the development period of an untreated control. 100% means that none of the flies has hatched, 0% means that all of the flies hatched normally.

In this test, at an exemplary active compound concentration of 2 500 ppm, the compounds from Preparation Examples Nos. 1-77, 1-78 and 1-79 show that no flies hatched.

What is claimed is:

1-13. (Canceled)

14. A compound of formula (I) 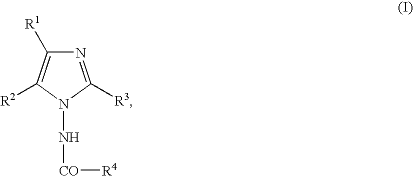
in which
R¹ and R² independently of one another represent hydrogen, alkyl, alkoxyalkyl, alkylthioalkyl; represent optionally substituted cycloalkyl; or represent optionally substituted heterocyclyl; or R¹ and R² together with the carbon atoms to which they are attached represent an optionally substituted mono- or bicyclic, carbocyclic, or heterocyclic group, and
R³ and R⁴ independently of one another represent optionally substituted aryl or hetaryl.

15. A compound of formula (I) according to claim 14 in which
R¹ and R² independently of one another represent hydrogen, C₁-C₆-alkyl, C₁-C₆-alkoxy-C₁-C₃-alkyl, or C₁-C₃-alkylthio-C₁-C₃-alkyl; or represent optionally C₁-C₃-alkyl-substituted C₃-C₇-cycloalkyl, which may optionally be interrupted by one or two oxygen and/or sulphur atoms; or R¹ and R² together with the carbon atoms to which they are attached represent optionally C₁-C₃-alkyl-substituted C₃-C₄-alkanediyl in which two carbon atoms that are not directly adjacent optionally form a further cyclic structure that is optionally interrupted by one oxygen and/or sulphur atom,
R³ represents phenyl or hetaryl, each of which is optionally substituted once or twice by identical or different C₁-C₄-alkyl, C₁-C₄-alkoxy, halogen, C₁-C₄-haloalkyl, C₁-C₄-haloalkoxy, cyano, nitro, C₁-C₄-alkylsulfonyl, C₁-C₄-halo-alkylsulfonyl, thioamide, or tetrazole substituents, and
R⁴ represents phenyl that is optionally substituted from one to four times by identical or different C₁-C₄-alkyl, C₁-C₄-alkoxy, C₁-C₄-alkoxy-C₁-C₄-alkoxy, halogen, C₁-C₄-haloalkyl, C₁-C₄-haloalkoxy, or cyano substituents and in which two adjacent carbon atoms optionally form a five- or six-membered carbocycle that is optionally interrupted by one or two oxygen atoms; or
represents hetaryl.

16. A compound of formula (I) according to claim 14 in which
R¹ and R² independently of one another represent hydrogen, C₁-C₅-alkyl, C₁-C₃-alkoxy-C₁-C₃-alkyl, or C₁-C₃-alkylthio-C₁-C₃-alkyl; or represent C₃-C₆-cycloalkyl; or R¹ and R² together with the carbon atoms to which they are attached represent C₃-C₄-alkanediyl that is optionally substituted from one to three times by identical or different C₁-C₃-alkyl substituents and in which two carbon atoms not directly adjacent optionally form a further cyclic structure that is optionally interrupted by an oxygen or sulphur atom,
R³ represents phenyl, pyridyl, thienyl, furyl, or pyrimidinyl, each of which is optionally substituted once or twice by identical or different methyl, fluorine, chlorine, bromine, iodine, cyano, nitro, methoxy, trifluoromethyl, trifluoromethoxy, sulfonylmethyl, sulfonyltrifluoromethyl, thioamide, or tetrazole substituents, and
R⁴ represents phenyl that is optionally substituted from one to four times by identical or different methyl, ethyl, fluorine, chlorine, bromine, trifluoromethyl, methoxy, trifluoromethoxy, ethoxymethyl ether, or cyano substituents and in which two adjacent carbon atoms optionally form a five- or six-membered carbocycle that is optionally interrupted by one or two oxygen atoms.

17. A compound of formula (I) according to claim 14 in which
R¹ represents hydrogen or methyl,
R² represents hydrogen, methyl, ethyl, n- or isopropyl, n-, iso- or s-butyl, cyclopentyl, or cyclohexyl; or R¹ and R² together with the carbon atoms to which they are attached represent C₃-C₄-alkanediyl that is optionally substituted once or twice by methyl or represent C₄-alkanediyl in which two carbon atoms not directly adjacent optionally form a further cyclic structure that is optionally interrupted by an oxygen atom,
R³ represents phenyl that is optionally substituted once or twice by identical or different methyl, fluorine, chlorine, bromine, iodine, nitro, cyano, methoxy, or trifluoromethyl substituents, and
R⁴ represents phenyl that is optionally substituted from one to four times by identical or different methyl, ethyl, fluorine, chlorine, bromine, trifluoromethyl, methoxy, trifluoromethoxy, 2-methoxyethoxy, or cyano substituents and in which two adjacent carbon atoms optionally form a five- or six-membered carbocycle that is optionally interrupted by one or two oxygen atoms.

18. A process for preparing a compound of formula (I) according to claim 14 comprising reacting a compound of formula (II) 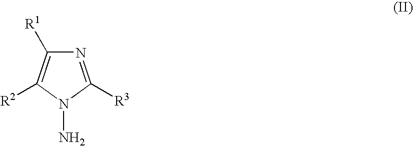
in which R¹, R² and R³ are as defined for formula (I) of claim 14, with a compound of formula (III)
R⁵—COR⁴ (III)
in which
R⁴ is as defined for formula (I), and
R⁵ represents halogen or hydroxyl in the presence of a diluent and an acid acceptor and/or a carboxylic-acid-activating reagent (coupling reagent) and optionally in the presence of a basic reaction auxiliary.

19. A compound of formula (II) 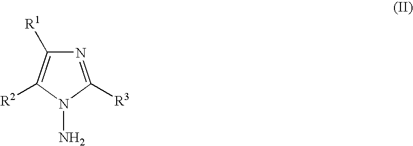
in which
R¹ and R² independently of one another represent hydrogen, alkyl, alkoxyalkyl, alkylthioalkyl; represent optionally substituted cycloalkyl; or represent optionally substituted heterocyclyl; or R¹ and R² together with the carbon atoms to which they are attached represent an optionally substituted mono- or bicyclic, carbocyclic, or heterocyclic group, and
R³ represents optionally substituted aryl or hetaryl, with the exception of 1-amino-2-phenylimidazole, 1-amino-2-(4-chlorophenyl)-imidazole, 1-amino-2-(4-methoxyphenyl)imidazole and 1-amino-2-(3,4-dimethoxyphenyl)imidazole.

20. A compound of formula (II) according to claim 19 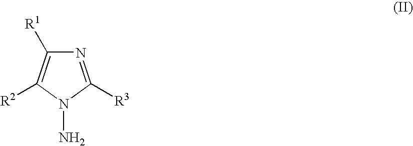
in which
R¹ and R² independently of one another represent hydrogen, C₁-C₆-alkyl, C₁-C₆-alkoxy-C₁-C₃-alkyl, or C₁-C₃-alkylthio-C₁-C₃-alkyl; or represent optionally C₁-C₃-alkyl-substituted C₃-C₇-cycloalkyl, which may optionally be interrupted by one or two oxygen and/or sulphur atoms; or R¹ and R² together with the carbon atoms to which they are attached represent optionally C₁-C₃-alkyl-substituted C₃-C₄-alkanediyl in which two carbon atoms that are not directly adjacent optionally form a further cyclic structure that is optionally interrupted by one oxygen and/or sulphur atom, and
R³ represents phenyl or hetaryl, each of which is optionally substituted once or twice by identical or different C₁-C₄-alkyl, C₁-C₄-alkoxy, halogen, C₁-C₄-haloalkyl, C₁-C₄-haloalkoxy, cyano, nitro, C₁-C₄-alkylsulfonyl, C₁-C₄-haloalkylsulfonyl, thioamide, or tetrazole substituents,
with the exception of 1-amino-2-phenylimidazole, 1-amino-2-(4-chlorophenyl)-imidazole, 1-amino-2-(4-methoxyphenyl)imidazole and 1-amino-2-(3,4-dimethoxyphenyl)imidazole.

21. A compound of formula (IV) 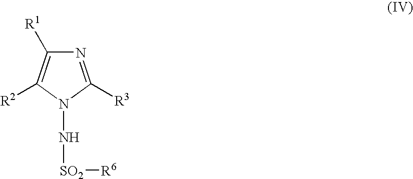
in which
R¹ and R² independently of one another represent hydrogen, alkyl, alkoxyalkyl, alkylthioalkyl; represent optionally substituted cycloalkyl; or represent optionally substituted heterocyclyl; or R¹ and R² together with the carbon atoms to which they are attached represent an optionally substituted mono- or bicyclic, carbocyclic, or heterocyclic group,
R³ represents optionally substituted aryl or hetaryl, and
R⁶ represents alkyl or optionally substituted phenyl.

22. A compound of formula (IV) according to claim 21 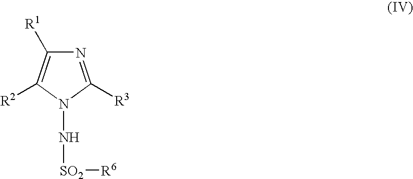
in which
R¹ and R² independently of one another represent hydrogen, C₁-C₆-alkyl, C₁-C₆-alkoxy-C₁-C₃-alkyl, or C₁-C₃-alkylthio-C₁-C₃-alkyl; or represent optionally C₁-C₃-alkyl-substituted C₃-C₇-cycloalkyl, which may optionally be interrupted by one or two oxygen and/or sulphur atoms; or R¹ and R² together with the carbon atoms to which they are attached represent optionally C₁-C₃-alkyl-substituted C₃-C₄-alkanediyl in which two carbon atoms that are not directly adjacent optionally form a further cyclic structure that is optionally interrupted by one oxygen and/or sulphur atom,
R³ represents phenyl or hetaryl, each of which is optionally substituted once or twice by identical or different C₁-C₄-alkyl, C₁-C₄-alkoxy, halogen, C₁-C₄-haloalkyl, C₁-C₄-haloalkoxy, cyano, nitro, C₁-C₄-alkylsulfonyl, C₁-C₄-haloalkylsulfonyl, thioamide, or tetrazole substituents, and
R⁶ represents alkyl or optionally substituted phenyl.

23. A compound of formula (III) 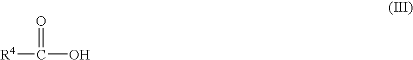
in which
R⁴ represents

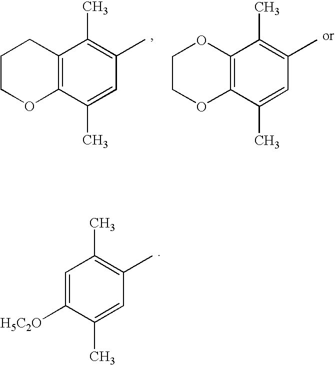

24. A pesticide comprising one or more compounds of formula (I) according to claim 14 and one or more extenders and/or surface-active substances.

25. A method of controlling animal pests comprising causing a compound of formula (I) according to claim 14 to act on a pest and/or its habitat.

26. A process for preparing pesticides comprising mixing a compound of formula (i) according to claim 14 with one or more extenders and/or surface-active substances.
